(19) United States
(12) Patent Application Publication
(10) Pub. No.: US 2026/0266733 A1
(43) Pub. Date: Sep. 10, 2026

(54) SYSTEM AND METHOD FOR ANALYZING SPECTRAL DATA USING A VALVED NON-THERMAL PLASMA REACTOR

(71) Applicant: Vionix Biosciences Inc., Belmont, CA (US)

(72) Inventors: Purshotam Rajani, Clayton, NC (US); Vivek Wadhwa, Belmont, CA (US)

(73) Assignee: Vionix Biosciences Inc., Belmont, CA (US)

(21) Appl. No.: 19/664,341

(22) Filed: Apr. 30, 2026

Related U.S. Application Data
(63) Continuation-in-part of application No. 19/562,004, filed on Mar. 10, 2026.
Application No. 19/562,004 is a continuation-in-part of application No. 19/427,370, filed on Dec. 19, 2025.
Application No. 19/427,370 is a continuation-in-part of application No. 19/388,299, filed on Nov. 13, 2025.
Application No. 19/388,299 is a continuation-in-part of application No. 19/374,579, filed on Oct. 30, 2025.
Application No. 19/374,579 is a continuation-in-part of application No. 19/347,104, filed on Oct. 1, 2025, now Pat. No. 12,651,650.
Application No. 19/347,104 is a continuation-in-part of application No. 18/828,382, filed on Sep. 9, 2024, now Pat. No. 12,437,842.
(60) Provisional application No. 63/541,177, filed on Sep. 28, 2023.
(60) Provisional application No. 63/854,206, filed on Jul. 30, 2025.
(60) Provisional application No. 63/855,046, filed on Jul. 31, 2025.
(60) Provisional application No. 63/870,459, filed on Aug. 26, 2025.
(60) Provisional application No. 63/870,464, filed on Aug. 26, 2025.

(51) Int. Cl. G01N 21/73; G01N 21/71

(52) U.S. Cl. CPC G01N 21/73; G01N 21/714; G01N 2201/129

(57) ABSTRACT

A system provides for an ability to automatically identify one or more chemical components of a sample, based on analysis of spectral data by at least one artificial intelligence module. The artificial intelligence module is able to be trained on a plurality of spectral data samples having known concentrations of individual chemicals and elements. The system is further operable to calculate the correlations between spectral data samples of varying concentrations and predict the concentration of the one or more chemical components of the sample.

### CROSS-REFERENCE TO RELATED APPLICATIONS

This application is related to and claims priority from the following U.S. patents and patent applications: this application is a continuation-in-part of U.S. patent application Ser. No. 19/562,004, filed Mar. 10, 2026, which is a continuation-in-part of U.S. patent application Ser. No. 19/427,370, filed Dec. 19, 2025, which is a continuation-in-part of U.S. patent application Ser. No. 19/388,299, filed Nov. 13, 2025, which is a continuation-in-part of U.S. patent application Ser. No. 19/374,579, filed Oct. 30, 2025, which is a continuation-in-part of U.S. patent application Ser. No. 19/347,104, filed Oct. 1, 2025, which is a continuation-in-part of U.S. patent application Ser. No. 18/828,382, filed Sep. 9, 2024, which claims priority from and the benefit of U.S. Provisional Patent Application No. 63/541,177, filed Sep. 28, 2023. U.S. patent application Ser. No. 19/347,104 further claims priority from and the benefit of U.S. Provisional Patent Application No. 63/854,206, filed Jul. 30, 2025, U.S. Provisional Patent Application No. 63/855,046, filed Jul. 31, 2025, U.S. Provisional Patent Application No. 63/870,459, filed Aug. 26, 2025, and U.S. Provisional Patent Application No. 63/870,464, filed Aug. 26, 2025. Each of the above documents is incorporated herein by reference in its entirety.

### BACKGROUND OF THE INVENTION

### 1. Field of the Invention

The present invention relates to spectroscopic analysis using a valved non-thermal plasma reactor providing for enhanced accuracy compared to non-thermal plasma reactors utilizing continuous flow.

### 2. Description of the Prior Art

It is generally known in the prior art to provide spectroscopic analysis for various substances in order to determine a chemical composition of those samples. Furthermore, it is known to provide analysis of substances using non-thermal plasma reactors which require a continuous flow of a substance through a reactor.

Prior art patent documents include the following:

US Patent Pub. No. 2021/0231597 for Fluid property sensor and fluid particle sensor by inventors Emokpae et al., filed Jan. 22, 2021 and published Jul. 29, 2021, discloses a method, system and apparatus for sensing fluids. A fluid sensor is configured to analyze a fluid utilizing impedance spectroscopy. Capacitive impedance of fluids is sensed and measured. Inductive impedance of suspended particles in fluids is measured. An electrochemical fingerprint of the properties of the fluid or of the particles within the fluid is generated. Fluid analytics data is generated from sensor signal data of the fluids under test. Trainable artificial intelligence algorithms are used to generate fluid analytics data.

U.S. Pat. No. 11,698,370 for Home toilet system for monitoring urine components in real time while urination by inventors Goldman et al., filed Sep. 29, 2021 and issued Jul. 11, 2023, discloses a system for urine sample analysis, where the system may include one or more transmitters for transmitting radiation; one or more sensors that are configured to receive received radiation that passed through the urine sample and to generate detection signals indicative of an intensity of the received radiation at multiple frequencies; detaching elements that are configured to detach the one or more transmitters and the one or more sensors to a toilet bowl; and a processor that is configured to participate in the urine sample analysis for determining a content of the urine sample based on the detection signals.

US Patent Pub. No. 2023/0034263 for Compositions and methods for spatial profiling of biological materials using time-resolved luminescence measurements by inventors Zhao et al., filed Nov. 19, 2020 and published Feb. 2, 2023, discloses compositions, including products of manufacture and kits, and methods, for in situ spatial profiling of biological materials such as DNA, RNA and protein in cells, tissues, and organisms for investigating biology and for conducting biomarker/drug discovery and development, and for clinical pathology and diagnosis. In alternative embodiments, provided are compositions, including products of manufacture and kits, and methods, for spatially determining, visualizing or quantifying target biological materials comprising in situ staining of a biological sample with one or a plurality of probes that are labeled with light-emitting moieties that exhibit or are encoded with distinct luminescence lifetime (and, optionally, spectrum) characteristics; followed by time-resolved luminescence imaging, measurement and analysis.

US Patent Pub. No. 2022/0268751 for Apparatus and method for multimode analytical sensing of items such as food by inventors Farkas et al., filed Feb. 25, 2022 and published Aug. 25, 2022, discloses a multimode biological sample inspection apparatus and method. The apparatus includes an illumination hardware arrangement comprising transmission and sensing hardware configured to inspect a biological sample using at least two modes from a group comprising a fluorescence imaging mode, a reflectance imaging mode, a scattering imaging mode, and a Raman imaging mode, and processing hardware configured to operate the illumination hardware arrangement according to a protocol comprising inspection settings of the at least two modes. The processing hardware receives scan results from the illumination hardware arrangement and identifies attributes of the biological sample by constructing a multidimensional dataset comprising at least one spatial dimension and at least one spectral dimension from the scan results and analyzing the multidimensional dataset. The processing hardware is configured to employ the attributes of at least one biological sample and alter the protocol.

U.S. Pat. No. 10,209,178 for Optical system for parameter characterization of an element of body fluid or tissue by inventors Carvalho Sousa et al., filed Jan. 31, 2014 and issued Feb. 19, 2019, discloses a biophotonic device for the point-of-care, real-time, non-invasive determination of parameters with diagnostic relevance, in particular an optical system for parameter characterization of an element of body fluid or tissue comprising an optical device which comprises: a light source for emitting light onto the element; and a spectrometer for recording the spectrum of light from the element, said light from the element being of transmittance, reflectance or Raman scattering of the emitted light by said element; the optical system further comprising a data processing module configured to: convert the recorded spectrum by a conversion matrix into a standardized spectrum, wherein said conversion matrix has been obtained by calibrating the optical system spectrum response against a spectrum reference; pre-process the converted spectrum; correlate, for parameter quantification, the converted pre-processed spectrum with pre-obtained spectral bands for each parameter; said spectrum being contained within uv-vis-nir wavelengths. Also methods of operating said system.

U.S. Pat. No. 11,650,195 for Iterative medical testing of biological samples by inventors Kaditz et al., filed Sep. 26, 2018 and issued May 16, 2023, discloses a system performing one or more magnetic resonance (MR) measurements on at least a portion of a biological life form. Moreover, the system quantitatively simulates an MR response of at least the portion of the biological life form, and compares the one or more MR measurements and the quantitative simulation to obtain a first test result. Next, the system determines one or more additional medical tests to perform. In response, the system accesses the biological sample in storage, and performs the one or more additional medical tests on at least a second portion of the biological sample to obtain one or more additional test results. Furthermore, the system computes a second test result based at least in part on the first test result and the one or more additional test results, where the second test result has an improved accuracy relative to the first test result.

US Patent Pub. No. 2021/0149361 for Real time monitoring of product purification by inventors Jungbauer et al., filed Apr. 4, 2017 and published May 20, 2021, discloses a method and device which allows in real-time the determination of concentration, purity and potency of a biological product during purification and/or concentration processes in order to intervene into the process, either for process control or real time release. The properties of the process stream are continuously monitored by at least two online sensors and with the aid of multivariate statistical analysis so that concentration, purity and potency is determined in real time.

US Patent Pub. No. 2022/0203407 for Sorting based on chemical composition by inventors Jungbauer et al., filed Mar. 16, 2022 and published Jun. 30, 2022, discloses systems and methods for classifying and sorting materials in order to produce a collection of materials that are composed of a particular chemical composition in the aggregate. The system may utilize a vision system and one or more sensor systems, which may implement a machine learning system in order to identify or classify each of the materials. The sorting is then performed as a function of the classifications.

US Patent Pub. No. 2023/0222654 for Machine learning systems and methods for assessment, healing prediction, and treatment of wounds by inventors Fan et al., filed Mar. 2, 2023 and published Jul. 13, 2023, discloses machine learning systems and methods for prediction of wound healing, such as for diabetic foot ulcers or other wounds, and for assessment implementations such as segmentation of images into wound regions and non-wound regions. Systems for assessing or predicting wound healing can include a light detection element configured to collect light of at least a first wavelength reflected from a tissue region including a wound, and one or more processors configured to generate an image based on a signal from the light detection element having pixels depicting the tissue region, determine reflectance intensity values for at least a subset of the pixels, determine one or more quantitative features of the subset of the plurality of pixels based on the reflectance intensity values, and generate a predicted or assessed healing parameter associated with the wound over a predetermined time interval.

U.S. Pat. No. 11,337,643 for Machine learning systems and techniques for multispectral amputation site analysis by inventors Fan et al., filed Aug. 24, 2020 and issued May 24, 2022, discloses certain aspects relating to apparatuses and techniques for non-invasive and non-contact optical imaging that acquire a plurality of images corresponding to both different times and different frequencies. Additionally, alternatives described herein are used with a variety of tissue classification applications including assessing the presence and severity of tissue conditions, such as necrosis and small vessel disease, at a potential or determined amputation site.

U.S. Patent Publication No. 2024/0328936 for an Apparatus, Systems and Methods for Use in Optical Gas Absorption Measurements by inventors Lawson, et al., filed Mar. 28, 2024 and published Oct. 3, 2024, is directed to an apparatus for optical gas detection and/or measurement in an absorption spectroscopy system comprising: a gas cell for containing a gas sample or calibration gas with at least one gas exchange port and at least one optical element for allowing transmission of electromagnetic radiation of a desired wavelength range in and out of the gas cell; and at least one source of converging, diverging or collimated electromagnetic radiation for transmitting electromagnetic radiation through a gas sample contained within the gas cell and towards at least one detector; said detector to monitor absorption of electromagnetic radiation for at least one absorption wavelength or wavelength range associated with at least one gas species, by detecting transmitted electromagnetic radiation that is not absorbed; at least one analyser for analysing an output signal from the at least one detector to measure a parameter of at least one gas species within the gas sample, characterised in that at least one dead volume within the spectrometer is filled with a transmissive filler material that reduces or eliminates the presence of at least one undesired gas species.

U.S. Pat. No. 5,046,846 for an Apparatus, Systems and Methods for Use in Optical Gas Absorption Measurements by inventors Ray, et al., filed Mar. 22, 1989 and issued Sep. 10, 1991, is directed to a method of analysis of and an apparatus for analysing a sample comprising the steps of: (A) examining at least one characteristic of a sample as a function of a variable parameter to obtain data, (B) normalizing the data with respect to a model, (C) making a comparison of the normalized data with reference data, and (D) determining from the comparison the quality of the sample. As a function of such determination of quality, control functions may be carried out with respect to a sample that was analyzed, such as a blend sample in a chemical processing plant.

U.S. Pat. No. 11,002,681 for Method for rapid raman spectra detection of a single bacterium or group of bacterial species by inventors Umapathy, et al., filed Jun. 4, 2018 and issued May 11, 2021, is directed to a method for rapid detection of a specific bacterial species. The method includes preparing a sample, irradiating the prepared sample with an electromagnetic radiation of specific wavelength, capturing the electromagnetic radiation scattered by the sample to obtain a Raman spectra and analyzing the Raman spectra to obtain a unique biochemical signature. The unique biochemical signature identifies a single bacterium within the sample. The method also provides for rapid detection of a group of bacterial species within the sample. The method takes about 1 minute to about 1 hour for the detection of specific bacterial species.

U.S. Pat. No. 10,914,683 for Methods and systems for identification of bacteria in biological fluid using raman spectroscopy and applications of same by inventors Mahadevan-Jansen, et al., filed Apr. 16, 2019 and issued Feb. 9, 2021, is directed to a method for identification and discrimination of bacteria and/or mutant bacterial strains in a biological fluid. The method includes illuminating the biological fluid with a beam of light; obtaining Raman data from light scattered from the illuminated biological fluid; and finding Raman signatures corresponding to each type of bacteria and/or mutant bacterial strains from the obtained Raman data, so as to identify and discriminate each type of bacteria and/or mutant bacterial strains in the biological fluid from the Raman signatures.

U.S. Pat. No. 12,055,495 for Diagnosis of disease using laser-induced breakdown spectroscopy and machine learning by inventors Guadiuso, et al., filed Jul. 16, 2021 and issued Aug. 6, 2024, is directed to diagnosing a pathology using LIBS and biological fluids, including focusing light on to a sample on a substrate to cause ablation of the sample and formation of a plasma, collecting optical emission from the plasma, and providing the optical emission to a spectroscopic acquisition component that provides information on spectral data of the plasma. The spectral data is provided to a machine learning algorithm to diagnose a pathology in the sample, where the algorithm is trained on a training set that includes spectral features in a difference spectrum derived from differences between a first LIBS optical emission spectrum collected from one or more samples of the biological fluid that have the pathology or known progress of the pathology and a second LIBS optical emission spectrum collected from one or more samples of the predetermined biological fluid that do not have the pathology or the known progress of the pathology.

U.S. Pat. No. 9,290,790 for Fluorescent method of detecting microorganisms resistant to a therapeutic agent by inventors Azevedo Pina Vaz, et al., filed Mar. 20, 2014 and issued Mar. 22, 2016, is directed to an antimicrobial susceptibility test made upon pure microbial cultures—a laboratory culture containing a single species of organism- or directly from biological samples uncultured. Some preferred embodiments do not require the use a pure culture obtained from a biological product which would otherwise take at least 24 hours for the microorganisms to grow-so, is performed an antimicrobial susceptibility test directly from uncultured biological samples like urine, blood cultures or water by fluorescence analysis.

U.S. Pat. No. 11,079,279 for Diagnosis method using laser induced breakdown spectroscopy and diagnosis device performing the same by inventors Pyun, et al., filed Apr. 30, 2020 and issued Aug. 3, 2021, is directed to a diagnostic method using laser induced breakdown spectrum analysis and a diagnostic device for performing the same. The diagnostic method may include projecting a pulsed laser to a suspicious specimen, obtaining first spectrum data on the light collected from the suspicious specimen, projecting the pulsed laser to a non-diseased specimen, obtaining second spectrum data on the light collected from the non-diseased specimen, and determining whether a disease is present in the suspicious specimen from a comparison value of the first spectrum data and the second spectrum data using an artificial neural network.

U.S. Patent Publication No. 2018/0098726 for Laser-based disease diagnosis system by inventors Pyun, et al., filed Jun. 28, 2017 and published Apr. 12, 2018, is directed to an aesthetic or medical laser system. The laser system includes: a spectrometer configured to measure a spectrum of light which is generated when body tissue is irradiated with a laser for beauty or medical care; a spectrum data comparison unit configured to compare the spectrum of the generated light measured by the spectrometer and a disease diagnosis reference spectrum data DB; and a disease diagnosis unit configured to determine whether there exists a disease in the body tissue based on a result of comparing by the spectrum data comparison unit.

U.S. Pat. No. 7,847,934 for Method for correcting spectral interference in ICP emission spectroscopy (OES) by inventor Neitsch, filed Jul. 2, 2008 and issued Dec. 7, 2010, is directed to a method for correcting spectral interference in a spectrum which is determined using an inductively coupled plasma spectrometer (ICP) for analysing element contents of a liquid or gaseous sample, comprising the following steps: recording the spectrum of a matrix solution containing all spectrally interfering components, which are also contained in the sample, in a first concentration; recording the spectrum of the matrix solution in at least one dilution of the first concentration; regressing the signal intensities obtained in steps a. and b. against the concentration for a number of wavelength positions; calibrating the spectrometer, background correction using the values determined from the regression in step c. and determining the calibration function c-f(I); recording the sample spectrum using at least one analyte which is contained therein; determining the concentration of the spectrally interfering components in the sample using the results obtained in step c. for wavelength positions at which no line of the analyte of the sample is present; and determining the sample signal which is characteristic of the analyte concentration by forming the difference between the spectrum from step e. and the calculated matrix spectrum in a dilution which was calculated in step f., wherein the calibration function c-f(I) is used.

U.S. Pat. No. 9,076,039 for Probabilistic identification of solid materials in hyperspectral imagery by inventors Halper, et al., filed Dec. 27, 2012 and issued Jul. 7, 2015, is directed to systems, methods and computer program products, for identification of materials based on hyperspectral imagery. An example system comprises one or more processors, a memory, a library of spectral signatures, a receiver, a model generator, and a material identifier. The receiver module is configured to receive a first spectral signature corresponding to a region of interest contained in the hyperspectral image. The model generator is configured to create a model search space including one or more model signatures based on the spectral signatures in the library, wherein each of the one or more model signatures approximate the first spectral signature. The material identifier is a material identifier configured to calculate a probability associated with a presence or absence of a material within the first spectral signature, based on the first spectral signature and the model search space and determine the presence or absence of the material in the region of interest based on the probability.

U.S. Pat. No. 11,373,839 for Method and system for component analysis of spectral data by inventors Hlavenka, et al., filed Feb. 3, 2021 and issued Jun. 28, 2022, is directed to a method and system where, responsive to irradiation of a charged particle beam, emission from sample is acquired in the form of spectral data. The spectral data is decomposed by a machine learning estimator to abundances and spectral components based on a character of the detector. Images showing compositional information of the sample are generated based on the abundances and the spectral components.

U.S. Pat. No. 11,933,740 for Method for the detection and quantification of nano or micro plastic particles by inventors Valsesia, et al., filed Jul. 30, 2021 and issued Mar. 19, 2024, is directed to a method for the detection of at least one nano or micro plastic particle comprised in a heterogeneous matrix material comprising the following steps: applying of at least one part of a heterogeneous matrix material comprising at least one nano or micro plastic particle onto at least a portion of a surface of a conductive support thereby forming a first layer onto said surface, irradiating of at least a portion of said first layer with at least one ion beam, thereby forming an irradiated layer, detecting of the at least one nano or micro plastic particle comprised in said irradiated layer by a detection method chosen from the group of Raman nanoscopic techniques, or infrared nanoscopic techniques, or charge dependent detection methods or combination thereof. The present invention allowed good detection of micro and nano plastic particles with high resolution and sensitivity.

U.S. Pat. No. 12,416,566 for Automated spectroscopic analysis of micron-scale microplastic particles with optical photothermal infrared spectroscopy by inventors Prater, et al., filed Jul. 11, 2023 and issued Sep. 16, 2025, is directed to a method and system where detection of microplastics is accomplished using a combination of techniques. A position-detection technique such as crossed-polarization detection, autofluorescence detection, or photothermal infrared imaging is used to determine the locations of microplastics in a sample. Infrared absorption can be detected at those locations to characterize the microplastics. In this way the microplastic content can be located and characterized more quickly and accurately than using conventional techniques.

U.S. Patent Publication No. 2023/0041820 for Detection of plastic microparticles by flow cytometry by inventors Franceschi, et al., filed Jul. 14, 2022 and published Feb. 9, 2023, is directed generally to the field of plastic microparticles. In particular, the present invention relates to the detection of plastic microparticles in a water-based sample. An embodiment of the present invention relates to a process for detecting and characterizing plastic microparticles in a water-based sample comprising the analysis of the sample by spectral flow cytometry. In accordance with the present invention, the process described herein may comprise the processing of the recorded flow cytometry data by a machine learning algorithm that can distinguish and categorize each particle based on its unique spectrum to characterize, for example, the plastic microparticles.

U.S. Pat. No. 6,562,386 for Method and apparatus for non-thermal pasteurization by inventors Rua, et al., filed May 7, 2021 and issued M7 13, 2003, is directed to a method and apparatus for at least partially sterilizing a liquid that has pathogens living in the liquid. The liquid comprising living pathogens is placed in a reaction volume, and a non-thermal plasma is generated within the reaction volume to thereby kill at least a portion of the pathogens within the liquid.

U.S. Pat. No. 10,676,353 for Systems and methods for processing gases by inventors Soane, et al., filed Oct. 15, 2019 and issued Jun. 9, 2020, is directed to a gas processing system for transforming a hydrocarbon-containing inflow gas into outflow gas products, where the system includes a gas delivery subsystem, a plasma reaction chamber, and a microwave subsystem, with the gas delivery subsystem in fluid communication with the plasma reaction chamber, so that the gas delivery subsystem directs the hydrocarbon-containing inflow gas into the plasma reaction chamber, and the microwave subsystem directs microwave energy into the plasma reaction chamber to energize the hydrocarbon-containing inflow gas, thereby forming a plasma in the plasma reaction chamber, which plasma effects the transformation of a hydrocarbon in the hydrocarbon-containing inflow gas into the outflow gas products, which comprise acetylene and hydrogen. The invention also includes methods for the use of the gas processing system.

### SUMMARY OF THE INVENTION

The present invention relates to spectroscopic analysis techniques, and more specifically to artificial intelligence-based spectroscopic analysis for multi-element and multi-macromolecule systems in analytes.

It is an object of this invention to provide automatic analysis of spectrographic test results to identify chemical compositions of sample analytes, especially fluids, in order to improve speed and accuracy of results.

In one embodiment, the present invention is directed to a system for identifying elements, molecules, and/or mixtures in a sample, including at least one reactor configured to receive a fluid sample; a vacuum pump operable to pump the fluid sample into the at least one reactor; a first valve positioned on an upstream side of the reactor; a second valve positioned on a downstream side of the reactor; a plurality of electrodes surrounding the reactor, configured to generate electric fields within the reactor to ionize the fluid sample into a non-thermal plasma; a spectrometer positioned proximate to the reactor, configured to generate absorption or emission spectral data for the fluid sample; and a processor in communication with the spectrometer configured to receive the absorption or emission spectral data and determine presence and/or concentration of one or more analytes in the fluid sample; wherein closing the first valve and the second valve causes the fluid sample to be contained in the at least one reactor, and wherein the fluid sample remains in the at least one reactor until the second valve is opened.

In another embodiment, the present invention is directed to a system for identifying elements, molecules, and/or mixtures in a sample, comprising: at least one reactor configured to receive a fluid sample; a first valve positioned on an upstream side of the reactor; a second valve positioned on a downstream side of the reactor; at least one electrode contacting a surface of the reactor or proximate to a surface of the reactor, configured to generate electric fields within the reactor to ionize the fluid sample into a non-thermal plasma; a spectrometer positioned proximate to the reactor, configured to generate absorption or emission spectral data for the fluid sample; at least one optical bridge coupling an exterior surface of the reactor with the spectrometer; and a processor in communication with the spectrometer configured to receive the absorption or emission spectral data and determine presence and/or concentration of one or more analytes in the fluid sample; wherein closing the first valve and the second valve causes the fluid sample to be contained in the at least one reactor, and wherein the fluid sample remains in the at least one reactor until the second valve is opened.

In yet another embodiment, the present invention is directed to a method for identifying elements, molecules, and/or mixtures in a sample, comprising: isolating a fluid sample within a reactor by closing a first valve on a first side of the reactor and a second valve on a second side of the reactor; applying power to a plurality of electrodes surrounding the reactor to create a non-thermal plasma within the fluid sample; transmitting optical emissions via at least one optical bridge to a spectrometer; generating time-resolved spectral data for the fluid sample over a target integration time; and processing the time-resolved spectral data to determine presence and/or concentration of one or more analytes in the fluid sample.

These and other aspects of the present invention will become apparent to those skilled in the art after a reading of the following description of the preferred embodiment when considered with the drawings, as they support the claimed invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates one system of the present invention.

FIG. 2A illustrates a subdivided spectral graph of the present invention.

FIG. 2B illustrates a subdivided spectral graph of the present invention.

FIG. 3 illustrates the composition of the intensity of an example measured wavelength.

FIG. 4 illustrates a chart of intensity versus wavelength for analysis of bidistilled water at different temperatures and pressures generated by the present invention.

FIG. 5 illustrates a chart of intensity versus wavelength for analysis of molecular biology grade (MBG) water at different temperatures and pressures generated by the present invention.

FIG. 6 illustrates a chart of intensity versus wavelength, comparing normalized peaks of bidistilled and molecular biology grade (MBG) generated by the present invention.

FIG. 7 illustrates a chart of intensity versus wavelength, comparing normalized peaks of sodium chloride (NaCl), dissolved in bidistilled water at different concentrations, pressures, and plasma power conditions generated by the present invention.

FIG. 8 illustrates a zoomed-in intensity versus wavelength chart for sodium chloride at different pressures, concentrations, and plasma power conditions, focused around a peak at 589 nm generated by the present invention.

FIG. 9 illustrates a chart of intensity versus wavelength, comparing samples with dATP at different pressure, concentration, and plasma power conditions relative to a water baseline generated by the present invention.

FIG. 10 illustrates a chart of intensity versus wavelength, comparing samples with dATP at different pressure, concentration, and plasma power conditions relative to a water baseline, zoomed in around 360 nm, generated by the present invention.

FIG. 11 illustrates a chart of intensity versus wavelength, comparing samples with dATP, dCTP, dGTP, dTTP relative to a water baseline generated by the present invention.

FIG. 12 illustrates a chart of intensity versus wavelength, comparing samples with dATP, dCTP, dGTP, dTTP relative to a water baseline, zoomed in between 300 and 400 nm, generated by the present invention.

FIG. 13 illustrates a chart of intensity versus wavelength, comparing samples with acetone, ethanol, and MBG water generated by the present invention.

FIG. 14 illustrates a chart of intensity versus wavelength, comparing samples with acetone, ethanol, and MBG water, zoomed in between 350 and 650 nm, generated by the present invention.

FIG. 15 illustrates a chart of intensity versus wavelength, comparing samples with acetone and ethanol, zoomed in between 350 and 650 nm with the water baseline subtracted, generated by the present invention.

FIG. 16 illustrates a chart of intensity versus wavelength, comparing samples with acetone and ethanol, zoomed in between 350 and 650 nm normalized at a peak at 482 nm, generated by the present invention.

FIG. 17 illustrates a chart of intensity versus wavelength, comparing samples with acetone and ethanol, zoomed in between 465 and 500 nm, showing a peak unique to the ethanol signature at approximately 485 nm, generated by the present invention.

FIG. 18 illustrates a system with a plasma reactor including valves for static analysis of a fluid sample according to one embodiment of the present invention.

FIG. 19 is a schematic diagram of a system of the present invention.

### DETAILED DESCRIPTION

The present invention is generally directed to spectroscopic analysis utilizing a valved non-thermal plasma reactor providing for monitoring the temporal evolution of a plasma-excited sample over various integration times. The valved non-thermal plasma reactor provides for distinguishing among overlapping molecular signatures using an artificial intelligence module, providing improvements over prior art spectroscopy which depends upon continuous flow thermal plasma reactors.

It is an object of this invention to provide automatic analysis of spectrographic test results to identify chemical compositions of sample analytes, especially fluids, in order to improve speed and accuracy of results.

In one embodiment, the present invention is directed to a system for identifying elements, molecules, and/or mixtures in a sample, including at least one reactor configured to receive a fluid sample; a vacuum pump operable to pump the fluid sample into the at least one reactor; a first valve positioned on an upstream side of the reactor; a second valve positioned on a downstream side of the reactor; a plurality of electrodes surrounding the reactor, configured to generate electric fields within the reactor to ionize the fluid sample into a non-thermal plasma; a spectrometer positioned proximate to the reactor, configured to generate absorption or emission spectral data for the fluid sample; and a processor in communication with the spectrometer configured to receive the absorption or emission spectral data and determine presence and/or concentration of one or more analytes in the fluid sample; wherein closing the first valve and the second valve causes the fluid sample to be contained in the at least one reactor, and wherein the fluid sample remains in the at least one reactor until the second valve is opened.

In another embodiment, the present invention is directed to a system for identifying elements, molecules, and/or mixtures in a sample, comprising: at least one reactor configured to receive a fluid sample; a first valve positioned on an upstream side of the reactor; a second valve positioned on a downstream side of the reactor; at least one electrode contacting a surface of the reactor or proximate to a surface of the reactor, configured to generate electric fields within the reactor to ionize the fluid sample into a non-thermal plasma; a spectrometer positioned proximate to the reactor, configured to generate absorption or emission spectral data for the fluid sample; at least one optical bridge coupling an exterior surface of the reactor with the spectrometer; and a processor in communication with the spectrometer configured to receive the absorption or emission spectral data and determine presence and/or concentration of one or more analytes in the fluid sample; wherein closing the first valve and the second valve causes the fluid sample to be contained in the at least one reactor, and wherein the fluid sample remains in the at least one reactor until the second valve is opened.

In yet another embodiment, the present invention is directed to a method for identifying elements, molecules, and/or mixtures in a sample, comprising: isolating a fluid sample within a reactor by closing a first valve on a first side of the reactor and a second valve on a second side of the reactor; applying power to a plurality of electrodes surrounding the reactor to create a non-thermal plasma within the fluid sample; transmitting optical emissions via at least one optical bridge to a spectrometer; generating time-resolved spectral data for the fluid sample over a target integration time; and processing the time-resolved spectral data to determine presence and/or concentration of one or more analytes in the fluid sample.

Spectroscopic techniques are a quintessential method of analyzing chemical composition of analytes. Various spectroscopic techniques rely on different techniques to treat an analyte and rely on different phenomena of molecules in order to identify the analyte. For example, infrared (IR) spectroscopy involves emitting infrared radiation on an analyte and then detecting the absorbance of the radiation from the molecules within at different wavelengths. The vibrational modes of particular functional groups or bond types within the material tend to absorb infrared radiation at different wavelengths, allowing for determination of at least properties of one or more chemical components within the analyte. Most commonly, spectroscopic tests are performed for analytes with one or only a few unknown components, allowing for simpler, albeit not always very simple, investigation of the composition of a small number of chemicals. However, difficulty arises in these techniques where there are many components having different functional groups, especially where those different components are larger and have more functional components.

Difficulties arise, in part, because analytes with many types of molecules tend to produce a high density of peaks within the technique that are difficult for a human operator to accurately distinguish or determine whether higher peaks indicate multiple components with the same functional group or a single component having the group, but having high concentration. For complex analytes such as organic compounds and medical fluids, these issues have partially been resolved by utilizing a wide array of tests for the same sample types, where the variety of tests are able to detect the presence or absence of specific molecules or classes of molecules. For example, for blood analysis, a wide variety of tests, from enzymatic tests and flow cytometry, to Raman spectroscopy and beyond are used to provide a more comprehensive view of the blood sample. However, the use of this wide variety of test techniques also necessitates higher expense and longer time than is ideal for informing patients about potentially serious issues that require resolution. Most commonly, not all of these tests are capable of being performed in the doctor's office or hospital, extending the time even further to account for transport of samples and communication with an external lab. Therefore, a method is needed to simplify analysis of sample analytes to utilize a single device (or a relatively low number of devices) in order to reduce time and money required for analysis.

Optical emission spectroscopy (OES), specifically, is a widely used analytical technique to determine the chemical composition of a variety of elements and compounds, typically for determining the elemental composition of metals. In optical emission spectroscopy, spectral lines are emitted by every analyte (element or compound) and are unique to that analyte. The emission lines from the analyte then enter the spectrometer and become split into spectral wavelengths by a diffraction grating element in the spectrometer. The intensity of light for each spectral wavelength is then detected by a corresponding detector. A computer system then measures the intensity of the spectral lines in the spectrum to determine the concentration of each element in the analyte.

However, OES as a technique begins to struggle when it analyzes samples with many kinds of molecules. These samples tend to produce a high density of spectral emission wavelengths which make it difficult for a human operator to accurately distinguish whether higher intensities indicate multiple components with the same functional group or a single component at a higher concentration. Higher resolution optical emission spectrometers can overcome this problem by improving the spectral resolution. However, these optical spectrometers are more expensive and require extensive data collected over long periods of time to produce accurate results.

Pixel resolution and spectral resolution are two key parameters which determine spectrometer performance. Pixel resolution determines how the spectrum is quantized after light is measured by the spectrometer. A smaller pixel resolution means that the spectrum is quantized into a fewer number of “bins” or pixels and thus leads to less detail in the data from the spectrometer.

Due to non-idealities in the optics of a spectrometer, some shifts in wavelength may occur when using a spectrometer. While pixels of a spectrometer detector associated with the actual emission wavelengths will receive the greatest light intensity, pixels nearby the actual emission wavelengths will receive a lesser light intensity according to a falloff curve. Spectral resolution is then defined as the width of the collection of pixels which receive a light intensity above half of the peak light intensity. Spectral resolution is dependent on the pixel resolution but also depends on the optics of the spectrometer; for example, a larger slit in the spectrometer can lead to greater dispersion in the wavelength of light that reaches the light detector, and therefore a lesser spectral resolution. Increasing pixel resolution and spectral resolution typically requires expensive and specialized hardware that can be difficult to obtain. Therefore, a system is needed to accurately distinguish wavelengths from an analyte on a spectrum in a cost-effective manner with a lower pixel and spectral resolution.

Additionally, prior art spectroscopy methods typically rely on one or a few characteristic wavelengths or peaks for detection of the composition of an analyte. This approach does not account for other indicators of the presence of an analyte, such as a faint baseline rise or a dip at an unusual wavelength that correlates with analyte levels. By analyzing not just a single spectral line on a particular wavelength and analyzing patterns for detection of analytes at multiple wavelengths, the present invention provides for detection of analytes that traditional spectroscopy methods do not detect.

Current prior art bacterial detection methods also have several shortcomings. To detect bacteria, a lab most commonly performs polymerase chain reaction (PCR) or grows a bacterial culture, which takes at least a few hours and a few days, respectively. Furthermore, PCR and enzyme-linked immunosorbent assay (ELISA) identify bacteria through primers designed for a specific sequence of DNA of a bacterium in the case of PCR, and antibodies which are specific to a certain bacterial antigen in the case of ELISA. This means that PCR and ELISA are only operable to detect the few bacteria for which the corresponding primer or antibodies were introduced, requiring prior assumptions as to which bacteria may be present within the testing sample.

The bacterial detection method of the present invention, on the other hand, is high-throughput, low-cost, and provides results in a matter of minutes, allowing for faster and easier testing for bacteria. Due to its speed and cost, the present invention provides for emergent use cases for which prior art methods were unsustainable, for example routine screening for emerging infection in patients or continuous environmental monitoring. The present invention requires no reagents, such as the primers used for PCR or the antibodies used for ELISA, and therefore is operable to detect any type of bacteria in a sample no matter how the test is designed.

Current prior art per- and polyfluoroalkyl (PFAS) detection methods have several shortcomings. To detect PFAS, a lab most commonly utilizes liquid chromatograph-tandem mass spectrometry (LC-MS/MS). LC-MS/MS is a multi-step process that requires separating compounds (e.g., filtration) in a sample and then analyzing the mass-to-charge ratios. LC-MS/MS requires coupling liquid chromatography with tandem mass spectrometry, which yields both parent and fragment ion peaks, which is helpful for detecting complex mixtures and/or low-abundance compounds, such as PFAS in a biological fluid. While LC-MS/MS produces accurate results, the process is time consuming due to the multi-step process. Therefore, there exists an unmet need for a system operable to detect PFAS in a single-step process. The present invention is operable to detect PFAS via optical emission spectroscopy, which does not require filtration or combining various detection methods.

Prior art plasma reactors are also limited in that these reactors require continuous flow of a substance. While these prior art systems provide for high-throughput, there are often overlapping molecular signatures which cannot be distinguished. In particular, complex samples, such as biological or environmental samples, require extended, undisturbed integration times to isolate faint trace analytes or microplastics. Continuous flow plasma reactors do not provide for a molecule to be in the excitation zone of the reactor for extended time periods, hindering the ability for the system to accurately identify the molecule.

While static liquid plasma systems which do not involve a continuous flow of liquid through the system exist, the accuracy of these systems is often affected by high signal-to-noise ratios and inefficient optical coupling. One such example of a prior art system includes systems which utilize atmospheric pressure dielectric barrier discharges over open vessels.

Accordingly, there is a need for a valved non-thermal plasma reactor which provides for pressure regulation and enhanced optical coupling to capture temporally dynamic spectral emissions from a sample.

Referring now to the drawings in general, the illustrations are for the purpose of describing one or more preferred embodiments of the invention and are not intended to limit the invention thereto.

The present invention is operable to analyze the full spectrum graph of an analyte, which comprises one or more components. For the purposes of the present invention, “component” refers to an element, a compound, a molecule, microplastics (e.g., PFAS), a bacterium, a microorganism, and/or a condition associated with the analyte (e.g., a disease identified by a spectral signature in a blood analyte).

The analyte which is analyzed is operable to contain a highly complex combination of components, for example the present invention is operable to analyze the full spectrum graph of an organic sample analyte. Prior art spectroscopy methods typically rely on a single (or a few) spectral line(s) on a particular wavelength(s) to detect components in an analyte. The present invention advantageously allows for a holistic analysis because looking at the entire spectrum allows for detection of patterns that traditional spectroscopy methods do not detect.

The present invention includes a device (e.g., a computer, a smart phone, a tablet, a smart watch, etc.) including a processor and a memory operable to receive and analyze spectrographic test data from at least one spectrographic analysis device, especially an optical emission spectroscopy (OES) spectrometer. In one embodiment, the device includes at least one server in network communication directly with the at least one spectrographic analysis device, or through an intermediary device, such as a computer, tablet, smart phone, etc. In this embodiment, analysis is able to be done remotely, providing for additional computational power and sharing of spectroscopic data and test results. In one embodiment, the at least one server includes at least one quantum processor operable to assist in performing analysis on the spectrographic test data.

The present invention is operable to produce a multi-dimensional dataset of spectral data or other chemical analysis data generated by one or more spectrometers or other chemical analysis devices. This multi-dimensional data allows the AI model of the present invention to leverage diverse and orthogonal information from each dimension in the dataset to allow for a final classification of components in an analyte. In one embodiment, the dataset is multimodal, meaning that the system of the present invention is capable of combining different analysis modalities (e.g., UV spectra, Raman spectra, IR spectra, mass spec, etc.) whether produced by a single device or multiple different devices. This combination of multiple modalities allows the system to become more granular and precise in its evaluations. In addition to different spectral or other analysis data, the multi-dimensional dataset is further able to take into account additional modalities, including patient demographics (e.g., race, sex, age, etc.), patient medical history, patient vitals, location where a sample was taken, and/or other parameters, which often assist in providing additional context to measurements in a medical environment. In one embodiment, patient demographic data or other similar data is connected to the chemical analysis (e.g., spectroscopic data) as metadata in order to allow for association of the patient demographics with the particular samples. The multi-dimensional dataset is able to be generated at the chemical analysis devices or at the server itself as it combines data from different data sources.

The present invention is preferably able to be used for analysis of medical fluids (i.e., fluids generated by a human body or the body of another, non-human animal). Furthermore, the term “fluid” as used herein is used to refer to both gasses and liquids. Examples of medical fluids able to be analyzed by the present invention include blood, saliva, urine, sweat, lymph, amniotic fluid, synovial fluid, mucus, semen, and/or other medical fluids. The medical fluids are operable to be prepared using anticoagulants or standard calibration with a serum. However, the present invention is advantageously operable to analyze other analytes, such as organic fluids, organic compounds, environmental samples such as water or soil, and heavy metals and alloys. One skilled in the art will realize that optical emission spectroscopy (OES) is advantageous for other families of elements, molecules, and components, and will further realize that the analysis of the present invention can be applied to various other methods of spectroscopy, each of which having their different advantages. Therefore, the above list is shortened for brevity and clarity.

In one embodiment, the analytes are analyzed directly using a spectrometer. In another embodiment, the analytes are mixed or dissolved with a solvent, including by way of example and not limitation, water, nitric acid, formic acid, dichloromethane, hexane, methanol, acetonitrile, other volatile organic solvents, and any combination thereof. In a further embodiment, before analyzing an unknown analyte, the solvent to be used is itself analyzed to establish a baseline. Any spectrographic test data then collected is normalized against the established baseline of the solvent to create data only showing the emission spectra of the solute. By mixing the analyte with a solvent before analyzing it with a spectrometer, organic compounds are more easily analyzed by the spectrometer without unintentionally denaturing, transforming, or otherwise marring the compounds.

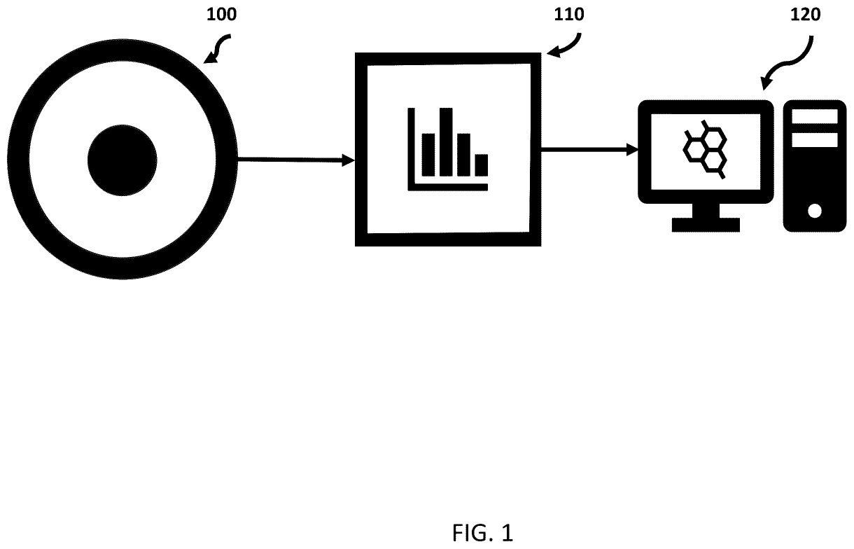

FIG. 1 illustrates one system of the present invention. The present invention comprises a chamber 100 configured to generate non-thermal plasma from the analyte and furthermore captures light from the plasma for analysis. The chamber is connected via optical cable to a spectrometer 110, which measures the light from the non-thermal plasma and generates spectrographic test data based on the light. The spectrographic test data is then sent to processor 120, which performs the aforementioned analysis on the spectrographic test data.

The present invention is preferably used with optical emission spectroscopy (OES). Using OES, the present invention generates plasma of the analyte to be analyzed and analyzes the light emitted from the plasma. In one embodiment, an optical cable (e.g. a fiber optic cable) is attached to a chamber where the plasma is generated to transmit the light to the optical emission spectrometer. In one embodiment, the plasma generated by the system is non-thermal plasma. In one embodiment, the chamber where the plasma is generated is subject to a vacuum to achieve zero air pressure or near-zero air pressure. Due to the characteristics of the hydrodynamic conditions in which these plasma chambers are able to operate, plasma is able to be generated and sustained at different energy levels, not requiring to evaporate all the fluid, allowing the detection of more sensitive and complex biomolecules, such as cholesterol or glucose that are otherwise broken down in other plasma-based analysis techniques, such as inductively charged plasma mass spectrometry (ICP-MS) or inductively charged plasma optical emission spectroscopy (ICP-OES), and molecular band emission of organic compounds (without decomposing them). The device is capable of achieving higher energy levels able to ionize even molecules, elements, and components with higher ionization energy (e.g., oxygen, nitrogen, etc.), which are not able to be detected in other spectrometry techniques (e.g., ICP-OES). This energy sweep ability, which allows to tune the ionization process for the detection of specific targets, is a fundamental feature of the present invention.

In one embodiment, the present invention is operable to use OES via a non-thermal plasma chamber. The non-thermal plasma chamber uses cold plasma and can operate at atmospheric pressure and ambient conditions, without needing bulky, high temperatures of ICP or lasers. Advantageously, directly exciting an unprocessed fluid sample in a cold plasma chamber, capturing the resultant optical emission spectrum, and using an artificial intelligence model trained to identify microplastic samples enables the present invention to use OES via a non-thermal plasma chamber.

The process carried out by the plasma reactors includes the transient transformation of a continuous stream of liquid into non-thermal plasma, a method comprising subjecting the liquid to an abrupt pressure drop, generating a liquid-gas biphasic stream, which is ionized by a controlled electric field inside a plasma reactor. This process has the unique ability to partially transform a continuous stream of water or other fluid into non-thermal plasma, with a sufficient amount of energy density to excite the atoms and molecules of the fluid, making them emit photons.

While some previous plasma chambers were focused on the use of the technology to sanitize water by breaking down potentially harmful agents in the water, the present invention is used for spectral analysis, instead of promoting disinfection mechanisms, with the process aimed at achieving a high degree of ionization for improved spectrographic detection.

One embodiment of the present invention includes a dielectric barrier discharge (DBD) plasma chamber that transforms a continuous sample flow (e.g., water flow, blood flow, serum plasma flow, etc.) into a biphasic stream of gas and liquid droplets at low pressure. This stream is then ionized by high-frequency electric fields produced by dielectric barrier electrodes, thereby producing plasma. In one embodiment, the frequency of the electric fields is 3.5 MHz. In one embodiment, the electrodes are positioned outside the reactor to prevent contamination. In a preferred embodiment, the electrodes are narrowly separated. The molecular and atomic photon band absorptions or emissions produced by the sample depend on the plasma conditions (e.g., the amount of energy used) and the composition of the sample stream.

The system of generating plasma, specifically non-thermal plasma, is described in further detail in U.S. patent application Ser. No. 18/805,927, which is incorporated herein by reference in its entirety.

Although the present invention is described with respect to OES with non-thermal plasma, the present invention is operable to be used in conjunction with many other types of spectrometry. In some cases and with specific analytes, other methods of spectrometry are preferable due to their specific mode of excitation. Examples of spectroscopic techniques whose results are able to be examined according to the present invention include ultraviolet-visual (UV/Vis) spectroscopy, near infrared (NIR) spectroscopy, infrared (IR) spectroscopy, Raman spectroscopy, mass spectrometry, nuclear magnetic resonance (NMR) spectroscopy (including continuous wave, Fourier-Transform, solid state NMR, magic-angle spinning NMR, etc.), X-ray spectroscopy, atomic emission spectroscopy (AES), Mossbauer spectroscopy, laser-induced breakdown spectroscopy (LIBS), and/or other forms of spectroscopic and spectrophotometric techniques. Likewise, while the present invention recites “emission intensity” when describing the output of a spectrometer, different methods of spectroscopy measure different variables with respect to wavelength, such as absorption of light. As such, emission intensity should be understood in the present invention to refer to the output of the type of spectrometry used.

In an alternative embodiment, the present invention is operable to use OES with inductively coupled plasma. In this embodiment, the plasma chamber includes a coil which is operable to generate an electromagnetic field. The electromagnetic field is then operable to ionize a gas, typically argon or nitrogen, into a plasma. The liquid-gas biphasic stream is then introduced to the plasma, which breaks down the sample and causes the sample's respective atoms to emit radiation at their respective characteristic wavelengths. This radiation is then measured with a spectrometer as described above.

In an alternative embodiment, the present invention is operable to use absorption spectroscopy. Rather than exciting the biphasic stream through cold plasma, inductively-coupled plasma, or lasers and measuring the resultant radiation, absorption spectroscopy focuses electromagnetic radiation (EMR) on a sample, preferably at a plurality of wavelengths, and measures the amount of EMR which is transmitted through the sample, thusly determining how much of the EMR is absorbed by the sample at the plurality of wavelengths. The system is operable to generate spectrographic test data based on these measurements. While both emission spectroscopy and absorption spectroscopy direct electromagnetic energy onto a sample, and although a measured emission and absorption spectrum are theoretically loosely related, both emission and absorption spectroscopy result in different interactions with the sample, and therefore different data which give different information about a sample.

Given that absorption spectroscopy aims to measure the EMR absorbed by a sample, rather than EMR emitted from a sample, a baseline is helpful in determining the amount that the intensity of measured transmitted EMR is lesser (and therefore, the amount of EMR absorbed by the sample). In one embodiment, the system is operable to measure a baseline spectrum with no sample placed within the reactor and compare the baseline with the measured spectrum of the sample. In a further embodiment, the system is operable to cache a recently measured baseline to increase the speed and ease at which the system can analyze samples. In another embodiment, the system includes a beamsplitter placed after the source of EMR, which splits the EMR between a channel including the sample, and a channel which does not include a sample. The system then measures the intensities of the EMR received through both channels, measuring the baseline spectrum and the measured spectrum at the same time. In another embodiment still, the AI module of the system is operable to infer the baseline spectrum from the measured spectrum, for example by detecting plateaus of high intensity within the measured spectrum.

Different methods of absorption spectroscopy exist, mainly dependent on the type of EMR directed onto the sample. With the appropriate source of EMR and a corresponding detector, the present invention is operable to use any of the following methods of absorption spectroscopy. Ultraviolet-visible (UV-vis) absorption spectroscopy utilizes EMR of ultraviolet and visible wavelengths. Other similar methods of absorption spectroscopy include X-ray absorption spectroscopy, infrared (IR) absorption spectroscopy, and microwave absorption spectroscopy, each leveraging their corresponding range of wavelengths. Nuclear magnetic resonance (NMR) spectroscopy measures the absorption of radio frequency (RF) EMR, typically within 4 to 900 MHz, by a sample which exhibits polarized nuclear spins under the influence of a magnetic field. Likewise, electron spin resonance (ESR) spectroscopy applies a similar concept by applying RF and/or microwave EMR to a sample under a magnetic field, but where the electrons of the sample exhibit spin rather than the nuclei. Since each of these methods produce a spectrum with characteristics representative of the makeup and/or structure of a sample, these methods are operable to be advantageously applied to the analysis of the present invention.

In one embodiment, the present invention is operable to capture temporal emission or absorption dynamics of the sample by measuring multiple spectra over a period of time after the introduction of EMR or other excitation of the sample. In this embodiment, the spectrometer preferably comprises a high-speed detector, such as an intensified charge-coupled device (ICCD), which is operable to capture a series of spectra in quick succession. In a further embodiment, when capturing temporal dynamics, the EMR or other excitation is conducted in short bursts, allowing the spectrometer to capture emission or absorption dynamics of the sample both as the EMR or other excitation is active and as the sample returns to a non-excited state.

The processor of the device includes an artificial intelligence (AI) engine, comprising at least one artificial intelligence model. The AI engine and its model are operable to perform analysis on the spectrographic test data and identify at least one component within the unknown analyte. The artificial intelligence engine and the model are further operable to determine the concentration of the at least one component within the unknown analyte. In one embodiment, the artificial intelligence engine is operable to output likelihoods associated with the at least one component within the unknown analyte, based, at least in part, on closeness of match to previous data or a model built based on analysis of previous data. The artificial intelligence engine and its model are described in further detail in U.S. Patent Publication No. 2025/0111900, which is incorporated herein by reference in its entirety.

The artificial intelligence model is particularly advantageous due to the holistic full-spectrum nature of its analysis. Traditional spectroscopic analysis takes into consideration only a few notable emission peaks of elements or compounds during identification. If these notable emission peaks are not identifiable, for example due to two compounds overlapping in emission at a specific wavelength, traditional spectroscopy typically mischaracterizes or fails to identify all the components of an unknown analyte. The artificial intelligence model of the present invention, however, is operable to identify and perform pattern matching on all features of emission spectra of components. Therefore, even if the most notable emission peak of a component overlaps with another component of the unknown analyte, the artificial intelligence model is operable to perform pattern matching on less notable features of the component to correctly identify it in the unknown analyte. These less notable features are often ignored or unnoticed in traditional spectroscopy. In this way, identification of all components of an analyte is improved over traditional spectroscopy, even in a complex sample with a crowded and overlapping emission spectrum.

Even if the emission spectra of two components do not exactly overlap, exact determination of their peak wavelengths (and thus determining the presence of the compounds) can prove difficult with conventional spectroscopy. If a spectrometer with lower wavelength resolution is used, there may not be enough clarity in the spectrographic test data to tell these peaks apart. This renders analysis of complex analytes using conventional spectroscopy impossible without prohibitively expensive high-resolution spectrometers, increasing the cost of sample analysis. Using the artificial intelligence model of the present invention, a spectrometer resolution high enough to discern two partially overlapping prominent emission peaks is not required; the artificial intelligence model is operable to perform pattern matching on less prominent features of the spectrographic test data, eliminating the need to discern the two partially overlapping peaks in the first place.

In one embodiment, the at least one artificial intelligence module is trained based on a plurality of prepared training data. Preferably, this training data corresponds with analytes with known chemical compositions, such that the at least one artificial intelligence module is able to correlate the output spectrographic test data with the known chemical composition to build a more accurate model. In this way, the system develops a characteristic profile for each component or set of components such that, under similar conditions and showing similar spectra, the system is able to identify particular components with a particular degree of certainty. The training data comprises spectral graphs, preferably multiple spectral graphs for each component. For the training data, each component is measured alone, or mixed or dissolved into a known solute as described above. In one embodiment, training of the artificial intelligence model employs early stopping based on validation loss and/or network regularization with dropout layers and/or L2 weight penalties. In one embodiment, training of the artificial intelligence model employs linear regression across one or more explanatory variables, non-linear regression across one or more explanatory variables, and/or combination or ensemble learning methods such as random forest regression and/or boosting algorithms.

In one embodiment, the at least one artificial intelligence module is operable to utilize vector embeddings to encode the entire emission spectrum into a feature vector for regression and/or classification. Spectral data is included in the vector embeddings such that the at least one artificial intelligence module compresses high-dimensional spectral data into a latent space and compares a library of spectra of known elements, molecules, and/or other components to an analyte. The at least one artificial intelligence engine is operable to use at least 50 spectral variables within the spectral data simultaneously. In one embodiment, the at least one artificial intelligence engine is operable to use 50 to 100 spectral variables simultaneously. In one embodiment the at least one artificial intelligence engine is operable to use 100 or more spectral variables simultaneously. As such, the at least one artificial intelligence module is operable to be tuned to analyze plasma emissions of complex organic analytes to detect, for example, microplastics. For example, the at least one artificial intelligence module is operable to generate a calibration curve for sub-sets of PFAS (e.g., perfluorooctanoic acid (PFOA)).

For each component, different measurements may vary testing parameters, for example the integration time of the analysis, the pressure of the chamber where plasma of the analyte is generated, the wavelength or intensity of the exciter which excites the analyte into plasma, the slit width of the spectrometer, plasma parameters, and/or the spectral resolution of the spectrometer. One skilled in the art will realize that many different parameters may be varied to produce more comprehensive results and training data, and the number of recited parameters has been limited for clarity and brevity. In a preferred embodiment, and especially when identifying the concentration of the components, the training data includes multiple measurements and spectral graphs for each component, wherein the concentration of the component is varied.

An example of such training data, which measures the emission lines of various elements, is the NIST Handbook of Basic Atomic Spectroscopic Data, version 1.1.3, dated November 2013. However, it is important to note that the present invention measures all components by generating plasma of the analyte, rather than the varying spectroscopy methods of the NIST data. Furthermore, training data for the purposes of the present invention should include multiple measurements for each component where the concentration of the component is varied.

In one embodiment, varying the plasma parameters includes monitoring the optical intensity or plasma impedance and adjusting power or gas flow during excitation. In one embodiment, excitation occurs via radio frequency (RF) discharge, dielectric barrier discharge (DBD), inductively coupled plasma, a laser-induced breakdown plasma, a microwave-induced plasma, and/or a glow discharge. In one embodiment, the system is operable to monitor the plasma optically or via electrical sensors and dynamically adjust the plasma parameters. To illustrate further, the system is operable to increase RF power and/or add a burst of oxygen when high organic content is detected and/or modulate the aerosol flow to prevent plasma dropouts. Varying the plasma parameters enables the system to detect microplastics present in part-per-billion concentration levels in an analyte sample as small as 1 milliliter or less.

In some embodiments, the system cycles through different plasma parameters to accentuate different parts of an emission spectrum. An artificial intelligence module is operable to fuse data obtained from an analyte after varying the different plasma parameters to improve accuracy of microplastic identification.

In one embodiment, the system is operable to generate a flame, arc, or spark from the sample to create measurable emissions. In another embodiment, the system is operable to generate emissions from the sample by focusing exciting electromagnetic radiation (EMR) at a plurality of wavelengths onto the sample. The system is then operable to measure the EMR emitted by the sample for each of the plurality of wavelengths of the exciting EMR.

In one embodiment, the training data further includes synthetic data (i.e., data not from actual tests) in order to expedite the model learning simple spectra of common elements, molecules, and other components. The use of an artificial intelligence module is especially important for analytes which comprise complex components that are unlikely to show simple peaks that are easily understood or reasoned out by a researcher (due to factors such as density of peaks and greater noise).

In one embodiment, the training data further includes measured emission lines of mixtures comprised of two or more components. The AI module is operable to be trained on the emission spectra of the mixtures and the known concentrations of the components which comprise the mixture. In one embodiment, the AI module is operable to analyze an unknown component and identify at least one mixture comprising the unknown component. By training the AI model on more complex substances, the AI model can learn how the emission lines of components comprising the mixture interact with one another, to more accurately and efficiently provide the composition of an unknown substance.

In one embodiment, the training data is operable to be preprocessed. Each raw spectrum is calibrated against known reference lines from, for example, a calibration lamp, and then is operable to be normalized by total emission or an internally defined reference line. In a further embodiment, the training data is further processed to identify the most relevant portions of spectrum. The processor is first operable to divide the spectrum graphs of the training data into at least one wavelength bin. For each wavelength bin, a correlation coefficient is then calculated. In an alternative embodiment, a correlation coefficient is calculated for each wavelength of the spectrum graphs.

The correlation coefficient describes how strongly the emission intensity correlates to the concentration of a component. This correlation coefficient is a numerical value which varies from −1 to 1, where −1 represents a strong negative correlation, 1 represents a strong positive correlation, and 0 represents no correlation. Therefore, if the correlation coefficient is 1, an increase in concentration of the component leads to a higher emission intensity. Likewise, if the correlation coefficient is −1, an increase in concentration of the component leads to a lower emission intensity. This negative correlation might occur if the component absorbs certain wavelengths of light, especially if it is dissolved in a solvent and absorbs some of the emissions of the solvent. One skilled in the art will realize there may be more reasons for observing negative correlations, especially considering non-thermal plasma generation is an emerging innovation. In one embodiment, the correlation coefficients are used to determine a confidence interval for the training data.

In one embodiment, the AI model is operable to be trained on the training data and learn patterns in emission spectra from the training data. In one embodiment, the AI model is of any type operable to learn features using a plurality of weights. The AI model is operable to learn the training data such that accuracy in determining the composition of a given analyte improves over time. The trained AI model allows quick identification of chemical composition at high levels of accuracy.

In one embodiment, the correlation coefficient is operable to be used to select the wavelength bins of the spectrum graphs of the training data which are used to train the artificial intelligence model. For example, if the absolute value of the correlation coefficient is above a certain value (e.g. p=0.4), the emission intensities for that bin are given to the artificial intelligence model for training, and other bins are discarded to reduce noise in the AI system.

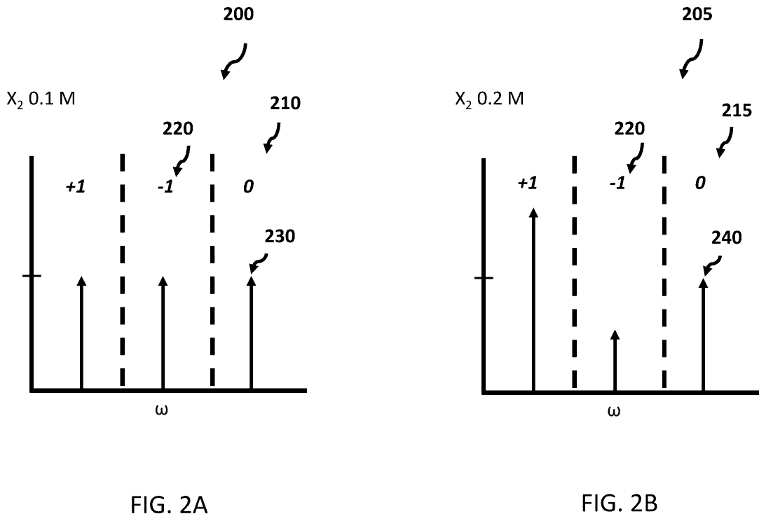

FIG. 2A and FIG. 2B illustrate the correlation coefficients for a hypothetical compound X₂. Two emission spectrum graphs 200, 205 are shown for different concentrations of the compound, at 0.1 moles on the left and 0.2 moles on the right. Three bins 210, 215 are created for each emission spectrum graph. The correlation coefficients 220 are shown for each bin. At 0.1 moles, the compound has equal emission intensities 230 at three points along the spectrum graph 200, however each emission intensity is contained within bins with different correlation coefficients (from left to right, +1, −1, and 0). As the concentration of the compound increases, as shown in spectrum graph 205, the expected emission intensities 240 change based on the correlation coefficients of their bins 215. For the +1 bin, the emission intensity increases when concentration increases; for the −1 bin, the emission intensity decreases when concentration increases; and for the 0 bin, there is no change in emission intensity with respect to concentration.

In one embodiment, the artificial intelligence engine is operable to generate correlation functions between the emission intensity and the concentration of a component. The artificial intelligence engine develops a line of best fit which follows a log-linear fit, a first degree, second degree, or n-degree polynomial fit, a linear fit, an exponential fit, or any other best-fit line. In a further embodiment, a separate line of best fit is developed for each of the wavelength bins of the spectrum graph, corresponding with the correlation coefficients of each bin.

In another embodiment, the artificial intelligence engine is operable to calculate a normalized spectral graph for a component. Physically, an electron jumping from a first electron orbital shell to a nearer second orbital shell emits energy with a wavelength of the difference of the radius of the first shell and the second shell. Therefore, the set of wavelengths emitted by a component are ordered by a definite set of ratios of their intensities.

The artificial intelligence engine is operable to calculate the normalized spectral graph R[λ] for a component. Normalization involves taking the maximum intensity value of the spectrum graph for the component and dividing the intensity of each wavelength of the spectral graph by the maximum intensity value, i.e. R[λ]=I[λ]/max(I[λ]). The benefit of R[λ] is that it is relatively constant for each component, no matter their concentration or the integration time of the measurement, making the normalized spectral graph especially useful for calculating the composition and concentrations of an unknown sample.

In one embodiment, the artificial intelligence engine is operable to decide whether to utilize the normalized spectral graph method or correlation coefficient method for determining composition and concentrations of an unknown sample, based on the number of components of the unknown sample. If the number of components in the unknown sample is large, the artificial intelligence engine is operable to utilize the normalized spectral graph method. Alternatively, if the number of components in the unknown sample is small, the artificial intelligence engine is operable to utilize the correlation coefficient method to determine the composition and concentrations of the components of an unknown sample.

In one embodiment, the trained artificial intelligence model is operable to receive an emission spectrum from an unknown analyte. The trained artificial intelligence model is operable to identify and perform pattern matching on all features of the emission spectrum to determine the composition of the unknown analyte, including which elements, compounds, and components are present in the analyte. Furthermore, the trained artificial intelligence model is operable to dynamically adjust spectrometer parameter settings including integration time, spectral resolution and/or entrance slit width to reduce noise and/or saturation in the emission spectrum.

In one embodiment, the trained artificial intelligence model is operable to determine certainty values for each of the identified components of the unknown analyte. In a further embodiment, the trained artificial intelligence model is operable to identify potential sets of components with associated certainty values. For example, the trained artificial intelligence model is operable to determine an exemplary analyte as comprising either components X and Y at 70% certainty or components X and Z at 30% certainty.

In a further embodiment still, based on the identified potential sets of components and their associated certainty values, the trained artificial intelligence model is operable to instruct the spectrometer to re-analyze specific wavelengths and/or ranges of wavelengths of the emission spectrum of the unknown analyte to differentiate between the potential sets of components. In one embodiment, the trained artificial intelligence model is additionally operable to modify one or more parameters of the spectrometer, including integration time, slit width, and/or spectral resolution, during re-analysis, based on identified differences between the potential sets of components.

Referring to the previous example, the trained artificial intelligence model has identified component X as a component of the analyte but still needs to determine whether component Y or Z is present. Accordingly, the trained artificial intelligence model is operable to determine wavelengths and/or ranges of wavelengths of the spectral graphs of components Y and Z which are most different from each other. The trained artificial intelligence model is then operable to instruct the spectrometer to re-analyze the unknown analyte with a higher integration time at the determined wavelengths and/or ranges of wavelengths to receive a more amplified and clearer emission spectrum of the wavelengths and/or ranges of wavelengths which are most different between components Y and Z. Using the amplified emission spectrum, the trained artificial intelligence model is operable to calculate updated certainty values for the potential sets of components. The trained artificial intelligence model is operable to recursively perform this process until the updated certainty value of one of the potential sets of components rises above a threshold. This process is advantageous and sometimes necessary to differentiate between complex biological components which have mostly identical but very slightly different emission spectra (owing to their composition of primarily carbon, hydrogen, nitrogen, etc.)

The trained artificial intelligence model is advantageous at determining the composition of an unknown analyte. When testing a trained artificial intelligence model, there are three relevant performance indicators: accuracy, precision, and recall. Accuracy is the ratio of correct predictions to all predictions made, precision is the ratio of correct positive identifications to all positives identified by the trained artificial intelligence model, and recall is the ratio of correct positive identifications to all positive cases in the testing dataset. The accuracy, precision, and recall of an exemplary trained artificial intelligence model according to the present invention, compared to the typical accuracy, precision, and recall of an inductively coupled plasma mass spectrometry (ICP-MS) system, is listed in Table 1 below:

| | | | | | ICP-MS | ICP-MS | ICP-MS |
|---|---|---|---|---|---|---|---|
| | Accuracy | Precision | Recall | | Accuracy | Precision | Recall |
| Analyte | (%) | (%) | (%) | Type | (%) | (%) | (%) |
| Acetonitrile | 95.38 | 98.1 | 77.11 | VOC | 95 | 95 | 85 |
| Butanol | 96.04 | 72.32 | 88.04 | VOC | 95 | 88 | 85 |
| Ethanol | 95.66 | 91.82 | 81.56 | VOC | 95 | 90 | 85 |
| Formaldehyde | 99.15 | 100 | 78.57 | VOC | 96 | 96 | 85 |
| Formic Acid | 97.83 | 91.84 | 70.31 | VOC | 94 | 90 | 80 |
| Methanol | 97.26 | 97.37 | 81.02 | VOC | 96 | 92 | 85 |
| Propanol | 99.15 | 76.67 | 92.00 | VOC | 95 | 90 | 90 |
| Tetrahydrofuran | 97.26 | 94.34 | 65.79 | VOC | 94 | 88 | 80 |
| Molybdenum | 99.40 | 100 | 92.60 | Heavy Metal | 96 | 94 | 85 |
| Chromium | 99.99 | 100 | 98.80 | Heavy Metal | 97 | 94 | 90 |
| Mercury | 99.67 | 99.94 | 98.80 | Heavy Metal | 97 | 94 | 88 |
| Fluoride | 99.99 | 100 | 98.90 | Non-metal | 95 | 90 | 80 |
| Titanium | 99.77 | 99.90 | 98.77 | Heavy Metal | 94 | 90 | 80 |
| Selenium | 99.50 | 99.76 | 97.70 | Heavy Metal | 95 | 92 | 85 |
| Antimony | 99.20 | 100 | 95.50 | Heavy Metal | 96 | 92 | 90 |
| Lead | 99.90 | 100 | 98.78 | Heavy Metal | 97 | 95 | 90 |

In an embodiment, the present invention is operable to determine the concentration of identified components in the unknown analyte. The trained artificial intelligence model, which has been trained on the training data of components with varying concentrations, is operable to determine the concentration of an identified component based on the spectral graph of the unknown analyte. For example, if an analyte contains 2 ppb of lead and 1 ppb of antimony, then after identifying the presence of lead and antimony as described above, the artificial intelligence model is operable to identify the concentrations of each comprising element from the spectral graph of the unknown analyte.

The trained artificial intelligence model is adept at determining the concentration of identified components, especially in light of the feature correlation and correlation coefficients of the training step. The percentage of correct concentration predictions of an analyte containing lead within different margins of error is listed below in Table 2:

| Error (%) | Accuracy (%) |
|---|---|
| 10 | 88.64 |
| 20 | 92.05 |
| 30 | 92.05 |
| 70 | 100 |

In another embodiment, the trained artificial intelligence model is operable to use the normalized spectral graphs R[λ] to find the composition of an unknown sample and the concentrations of the components which comprise the unknown sample. This calculation takes the form of an optimization problem, specifically a linear programming model with a set of variables, constraints, and a function to optimize.

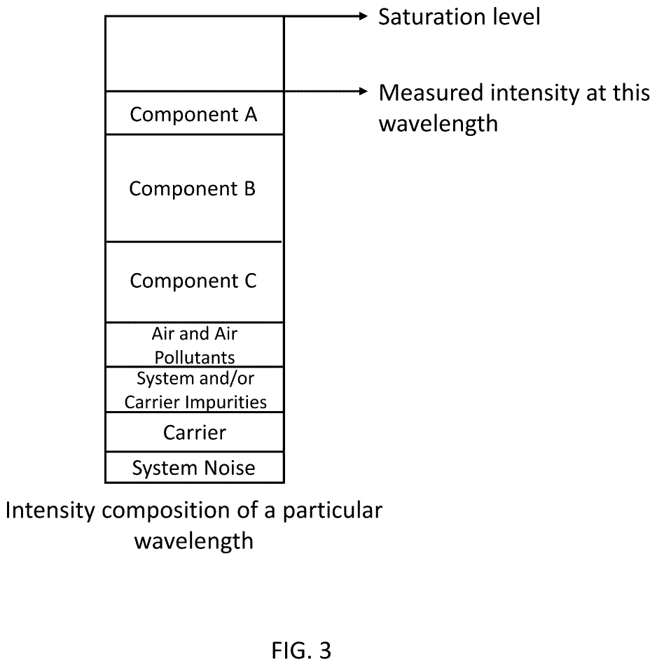

FIG. 3 shows the intensity composition of an individual wavelength of a measured spectral graph of an unknown sample. The measured intensity of the wavelength is a combination of light intensity contributions from a plurality of components (in FIG. 3, components A-C). Additionally, some undesired components are always present within wavelengths of a measured spectral graph, such as noise introduced to measurements by the spectrometer or other components of the system, a carrier of the analyte, impurities contained within the carrier or introduced by the system (e.g., through the reactor), and atmospheric air and pollutants and impurities within it.

Using the known normalized spectral graphs R[λ], the linear programming model is operable to solve for which components and concentrations minimize the overall difference between a calculated spectral graph for the unknown sample and the actual measured spectral graph. The linear programming model is further operable to quantify the contributions of undesired components (in FIG. 3, components other than components A-C) to remove the contributions of the undesired components from the calculation of the composition and concentrations of the components of the unknown sample.

Consider an unknown sample with N unknown components having intensities defined by I[n, λ], where n∈{0, 1, . . . , N−1}, and a measured spectral graph having intensities Iₜₒₜₐₗ[λ]. The N unknown components have associated normalized spectral graphs defined by R[n, λ]. Assuming all components are previously known by the system,

$${I}_{total} [ λ ] = {\sum }_{n = 0}^{N - 1} I [ n , λ ] .$$

However, the trained artificial intelligence model often is not trained on every component which is present in an unknown sample, and the measured spectrum graph does not typically represent an ideal sum of the spectral contributions of all components. A more apt constraint, assuming that the total intensities cannot be greater than the calculated sum of their parts, is

$${I}_{total} [ λ ] \leq {\sum }_{n = 0}^{N - 1} I [ n , λ ] .$$

The system is operable to optimize the set of N components, and their associated concentrations, such that

$${I}_{c a l c} [ λ ] = {\sum }_{n = 0}^{N - 1} I [ n , λ ]$$

matches as close as possible to the measured spectral graph Iₜₒₜₐₗ[λ]. In other words, the system minimizes the term Σ_{λ}Iₜₒₜₐₗ[λ]−I_{calc}[λ], i.e.,

$${\sum }_{λ} {I}_{total} [ λ ] - {\sum }_{λ} {\sum }_{n = 0}^{N - 1} I [ n , λ ] ,$$

where I[n, λ]=c[n]R[n, λ]. The system is operable to change the N components, and thus the set of normalized spectral graphs R[n, λ], as well as the concentrations of those components c[n], to solve the linear programming optimization problem and determine the elements, compounds, and/or components and their concentrations within the unknown sample.

One potential shortcoming of the term

$${I}_{c a l c} [ λ ] = {\sum }_{n = 0}^{N - 1} I [ n , λ ]$$

is that the light falloff of the spectrometer causes the intensity of each wavelength to be affected by the intensity of the light surrounding it. If, for example, a spectrometer measures a component which returns a single wavelength of emitted light, the actual measured spectral graph will resemble a function akin to a bell curve, and not a single wavelength emission component as expected ideally. This means that, referring back to FIG. 3, the intensity composition of a particular wavelength is often increased by the intensity of neighboring wavelengths and not just individual components.

In one embodiment, the system stores an impulse response of the spectrometer, which defines the light falloff of the spectrometer given a single impulse (like that of the single-wavelength example provided earlier). This impulse response is operable to be determined experimentally and/or provided by the manufacturer or specifications of the spectrometer. The system is then operable to calculate a convolution of the previous intensity compositions and the impulse response of the spectrometer to create a more accurate calculated spectral graph I_{calc}[λ]. Thus, in this embodiment,

$${I}_{c a l c} [ λ ] = ( {\sum }_{n = 0}^{N - 1} I [ n , λ ] ) * h [ λ ] ,$$

or alternatively

$${I}_{c a l c} [ λ ] = {\sum }_{k} ( ( {\sum }_{n = 0}^{N - 1} I [ n , k ] ) h [ λ - k ] ) ,$$

where h[λ] is the impulse response of the spectrometer. The system is then operable to use the result of the convolution I_{calc}[λ] to minimize the term Σ_{λ}Iₜₒₜₐₗ[λ]−I_{calc}[λ] as described above.

In one embodiment, the trained artificial intelligence model is operable to continue training while being used to identify unknown analytes. If one or more components are detected in an unknown analyte, the spectral graph and the identified relevant patterns and features of that analyte are categorized by the one or more components and added to the training data.

In one embodiment, the spectral graphs of analyzed unknown analytes are stored, either locally, on an external server, or in an external cloud environment. When the trained artificial intelligence model is updated, the present invention is operable to re-analyze the spectral graphs of previously measured unknown analytes to more accurately identify components, and/or to identify components that were previously not included in the training data. In this manner, the present invention is operable to not only leverage a continually trained model for new unknown analytes but is also operable to use an updated model for previously analyzed analytes.

In one embodiment, the artificial intelligence engine is operable to capture relevant patterns and features from a plurality of unknown analytes, even if those patterns and features cannot be categorized as a previously known component by the trained artificial intelligence model. Since the trained artificial intelligence model is trained on a sometimes limited set of training data, it is expected that the trained artificial intelligence model will encounter patterns and features which the training data does not include, and thus for which the trained artificial intelligence model cannot identify as a known component. The artificial intelligence engine is operable to identify reoccurring patterns and features within previously stored unknown analytes and identify spectral contributions which the trained artificial intelligence model is unable to correlate to a known component.

In one embodiment, identification of unknown spectral contributions is operable to be implemented using a clustering algorithm, such as K-means clustering. The artificial intelligence engine is further operable to generate a report if such a reoccurring pattern and/or feature is found, notifying the user that there is an unknown but often-occurring spectral contribution that could prompt further investigation. In a further embodiment, the artificial intelligence engine is operable to leverage the synthetic data and/or perform a simulation of ionized element and/or compound emissions to predict the one or more components responsible for an unknown spectral contribution.

In one embodiment, data produced by spectrometers or other chemical analysis devices associated with the present invention are able to be brought into a single cloud environment, optionally also including additional public dataset or other supporting datasets produced by third parties in order to aggregate and strengthen the training data. The cloud environment is further operable to continuously train the trained artificial intelligence model, incorporating new data collected by the spectrometers or other chemical analysis devices associated with the present invention. In this embodiment, the present invention is operable to download updated trained artificial intelligence models from the cloud environment, continually staying up-to-date with a model generated from the aggregated training data.

In this embodiment, all data brought into the cloud environment is anonymized, and in the case of the present invention being used in a medical environment, recording of data is operable to comply with local regulations regarding patient confidentiality. For example, recording of data to the cloud environment is operable to comply with the Health Insurance Portability and Accountability Act (HIPAA) if the present invention is used in the United States. In a further embodiment, the data brought into the cloud environment is geotagged at a non-identifying resolution based on the location at which the data was measured. For example, and not by limitation, geotagging is performed by postal code, by city, or via Internet Protocol (IP) address geolocation. In this manner, regional trends of identification are able to be aggregated as the present invention performs analyses, for example, for the spread of disease or the spread of environmental pollution.

In one embodiment, the present invention is operable to transmit collected data and download updated trained artificial intelligence modules through an integrated cell modem, via WI-FI, through a mesh network of other spectrometer devices, via LoRA-based communication, and/or via satellite connection.

In one embodiment, the report is able to include not only an identification of which components are present, but also an indication of the concentration of those components and/or a probability value associated with the identification to indicate a degree of certainty to which the model identified the reported component.

In one embodiment, the system is operable to produce interactive reports detailing what components are present in an analyte in different formats depending on the preference of the reader or consumer of the data.

In one embodiment, the at least one artificial intelligence module utilizes principal components analysis (PCA) and/or linear discriminant analysis (LDA) for feature compression in order to feed into the downstream machine learning (ML) model used by the present invention.

### Environmental Monitoring Applications

Monitoring of the environment is highly important, both when considering human habitability and the habitability of an area for animals, especially in areas of conservation. The status of water supplies and waterways and the presence of soil contaminants and air pollutants are key environmental factors to monitor. However, many areas exist where conventional environmental monitoring is not feasible due to remoteness or prohibitive costs. It is an object of this invention to provide for an environmental monitoring solution which is sufficiently accurate, yet operable to be used in remote areas and at a lower cost than existing solutions. In one embodiment, the present invention is operable to be used as an environmental monitoring system for the purpose of environmental monitoring and analysis.

In one embodiment, the environmental monitoring system is operable to be used manually, where samples are taken manually and analysis is manually initiated. In another embodiment, the environmental monitoring system is operable to collect samples and perform analysis autonomously.

When used autonomously, the present invention is operable to transmit generated reports automatically to an external server, which collects reports from a plurality of systems over a plurality of times. In one embodiment, the present invention is operable to generate an alert when the measured concentration of contaminants and/or pollutants rises above a threshold. In a further embodiment, the present invention is operable to generate an alert when the measured concentration is approaching a threshold (i.e., when the concentration is below, but close to, the threshold, and trending towards the threshold over time). In one embodiment, the artificial intelligence module of the present invention is operable to analyze reports over time and identify one or more hazardous conditions and/or predict one or more upcoming hazardous conditions. Hazardous conditions include, but are not limited to, concentrations of contaminants or pollutants rising above a dangerous level, concentrations of one or more contaminants or pollutants rising above a level harmful to one or more affected species, including humans, or concentrations of one or more contaminants or pollutants rising above a level detrimental to a specific application, for example the concentration of particulate matter rising above a threshold detrimental to growth of specific crops.

In one embodiment, the present invention is operable to be used for water monitoring, wherein a sample of water is the analyte. The analyte is operable to be water originating from a river, lake, ocean, or other body of water, rainwater, groundwater, or water from a municipal water supply. The system is operable to be used manually, deployed in a body of water for periodic autonomous analysis, or deployed inline in a water supply system for continuous autonomous analysis. In one embodiment, the present invention is waterproof and/or water resistant to the degree that it is able to be partially or fully submerged in a body of water to collect samples of the water autonomously.

The environmental monitoring system and its trained AI model, when used for water analysis, is operable to detect heavy metal contaminants, pesticides and/or herbicides, volatile organic compounds (VOCs), biological contaminants, microplastics (e.g., PFAS), and/or nutrients such as phosphorous or nitrogen, among others. In this embodiment, the AI module is trained based on a plurality of prepared training data, wherein at least some of the prepared training data corresponds to prepared training samples of the above elements and compounds, alone or in combination. The prepared training data is operable to be obtained with varying testing parameters, such as the integration time of the analysis, the slit width of the spectrometer, plasma parameters, and/or the spectral resolution of the spectrometer.

In one embodiment, the environmental monitoring system is operable to be used for analysis of soil, wherein a sample of soil is the analyte. The system is operable to be used manually or deployed in-field for periodic autonomous analysis. In one embodiment, the system is operable to be mounted to a remotely or autonomously controlled rover or unmanned vehicle.

When used for soil analysis, the environmental monitoring system and its trained AI module is operable to detect heavy metals, pesticides and/or herbicides, industrial solvents, petroleum hydrocarbons, polycyclic aromatic hydrocarbons, and/or radioactive wastes and materials, among others. In this embodiment, the AI module is trained based on a plurality of prepared training data, wherein at least some of the prepared training data corresponds to prepared training samples of the above elements and compounds, alone or in combination. The prepared training data is operable to be obtained with varying testing parameters, such as the integration time of the analysis, the slit width of the spectrometer, and/or the spectral resolution of the spectrometer.

In one embodiment, the environmental monitoring system is operable to be used for analysis of air quality. The system is operable to be used manually, deployed for periodic autonomous analysis, or deployed inline in a continuous flow of air or other gas for continuous autonomous analysis. In one embodiment, the system is operable to be mounted to a plane, helicopter, or remotely or autonomously controlled unmanned aerial vehicle (UAV).

The system is operable to be used for analysis of outside air quality or for analysis of air quality indoors. When used indoors, in one embodiment, the system is operable to communicate air quality information to a building management system (BMS). In an alternative embodiment, the system is operable to communicate with and/or control air quality control equipment and/or ventilation systems. For example, when the environmental monitoring system is used in a chemical laboratory, the system is operable to turn on a ventilation fan when a high concentration of dangerous gases is detected. In one embodiment, the present invention is operable to be used in a clean room to detect a high amount of particulate matter.

When used for air quality analysis, the environmental monitoring system and its trained AI module is operable to detect carbon monoxide, ozone, volatile organic compounds (VOCs), sulfur dioxide, ammonia, methane, nitrogen dioxide, heavy metals including lead, and/or particulate matter, among others. In this embodiment, the AI module is trained based on a plurality of prepared training data, wherein at least some of the prepared training data corresponds to prepared training samples of the above elements and compounds, alone or in combination. The prepared training data is operable to be obtained with varied testing parameters, such as the integration time of the analysis, the slit width of the spectrometer, and/or the spectral resolution of the spectrometer.

### INDUSTRIAL APPLICATIONS

Water and other fluids are heavily used in the refinery industry for cooling or circulation in a plant. Industrial fluid testing is important because fluid systems need to be able to resist corrosion and sedimentation formation. Traditional fluid testing typically comprises tracing sodium, transition metals, chloride and/or sulfate. However, the detection of trace levels of an analyte in an sample that has high ionic-strength matrices is extremely difficult. Therefore, there is a need for a system that is operable to accurately and quickly detect chemicals or molecules present in an analyte for industrial fluid systems.

In one embodiment, an industrial fluid or sample from an industrial water system is analyzed using a spectrometer for industrial fluid monitoring. In another embodiment, the industrial fluid is mixed or dissolved with a solvent, including by way of example and not limitation, water, nitric acid, formic acid, dichloromethane, hexane, methanol, acetonitrile, other volatile organic solvents, and any combination thereof.

In one embodiment, spectrographic data from the industrial fluid is passed to the AI module, comprising at least one artificial intelligence model. The AI engine and its model are operable to perform analysis on the spectrographic test data and identify at least one element or compound within the industrial fluid. In one embodiment, the AI engine is operable to determine a concentration of at least one element or compound within the industrial fluid.

In one embodiment, the AI module is trained based on a plurality of prepared training data, including data related to industrial fluid systems. The related prepared training data is operable to include data for elements or compounds at a variety of testing parameters, such as integration time of the analysis, pressure of the chamber where plasma of the fluid is generated, the slit width of the spectrometer, and/or the spectral resolution of the spectrometer.

In one embodiment, the trained AI model is operable to receive an emission spectrum from an industrial fluid. The trained AI model is operable to identify and perform pattern matching on all features of the emission spectrum to determine the chemical composition of the industrial analyte. Furthermore, the trained AI model is operable to dynamically adjust spectrometer parameter settings including integration time, spectral resolution and/or entrance slit width to reduce noise and/or saturation in the emission spectrum

Other industrial applications include monitoring the output of a chemical reactor for quality control, checking the composition of pharmaceutical mixtures during manufacturing, or even refinery process monitoring. While the present invention is preferably used with optical emission spectroscopy (OES), it is not necessarily limited to OES spectroscopy. The AI module of the present invention is operable to identify chemicals or compounds from other forms of spectrographic data, including IR absorption or Raman spectrum for an industrial fluid.

When used autonomously, the present invention is operable to transmit generated reports automatically to an external server, which collects reports from a plurality of systems over a plurality of time. In one embodiment, the present invention is operable to generate an alert when the measured concentration of contaminants and/or pollutants, such as trace sodium, transition metals, chloride and/or sulfate, rises above a threshold. In a further embodiment, the present invention is operable to generate an alert when the measured concentration is approaching a threshold (i.e., when the concentration is below, but close to, the threshold, and trending towards the threshold over time). In one embodiment, the artificial intelligence module of the present invention is operable to analyze reports over time and identify one or more hazardous conditions and/or predict one or more upcoming hazardous conditions, including sedimentation and/or corrosion.

In one embodiment, the at least one artificial intelligence module utilizes principal components analysis (PCA) and/or linear discriminant analysis (LDA) for feature compression in order to feed into the downstream machine learning (ML) model used by the present invention.

The system is operable to utilize a plurality of learning techniques including, but not limited to, machine learning (ML), artificial intelligence (AI), deep learning (DL), neural networks (NNs), artificial neural networks (ANNs), support vector machines (SVMs), transformers (e.g., generative pre-trained transformers), Markov decision process (MDP), random forest, multiple layer perceptron, recurrent neural networks, generalized adversarial networks, and/or natural language processing (NLP). The system is operable to use any of the aforementioned learning techniques alone or in combination.

Further, the system is operable to utilize predictive analytics techniques including, but not limited to, machine learning (ML), artificial intelligence (AI), neural networks (NNs) (e.g., long short term memory (LSTM) neural networks), transformers (e.g., generative pre-trained transformers), deep learning, historical data, and/or data mining to make future predictions and/or models. The system is preferably operable to recommend and/or perform actions based on historical data, external data sources, ML, AI, NNs, and/or other learning techniques. The system is operable to utilize predictive modeling and/or optimization algorithms including, but not limited to, heuristic algorithms, particle swarm optimization, genetic algorithms, technical analysis descriptors, combinatorial algorithms, quantum optimization algorithms, iterative methods, deep learning techniques, and/or feature selection techniques.

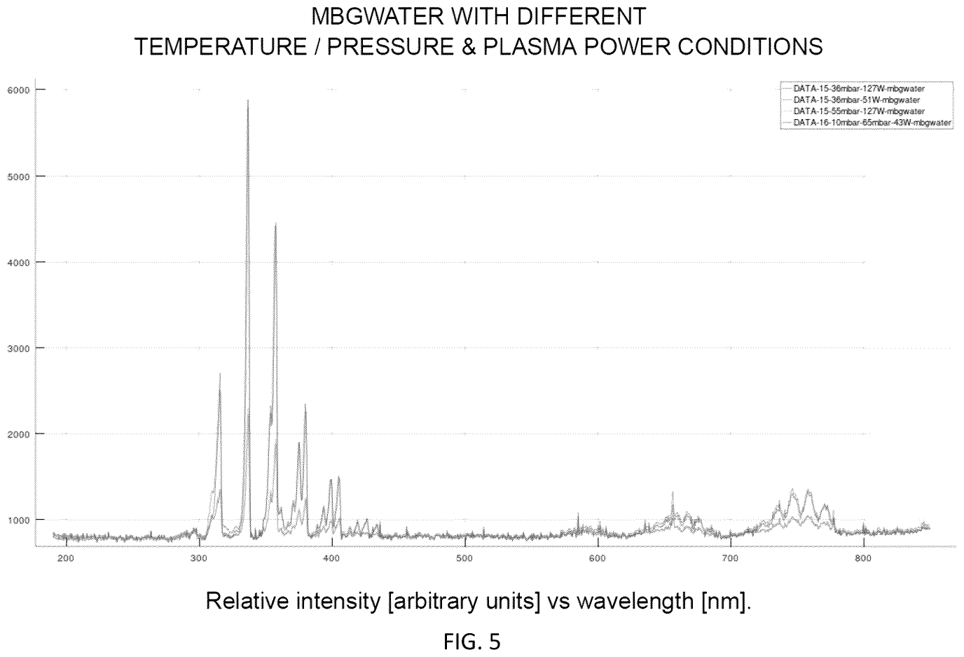

FIGS. 5-18 provide examples of spectral data able to be incorporated into the present invention to identify particular molecules and demonstration of the ability of such spectra to distinguish the presence and/or concentration of particular molecules.

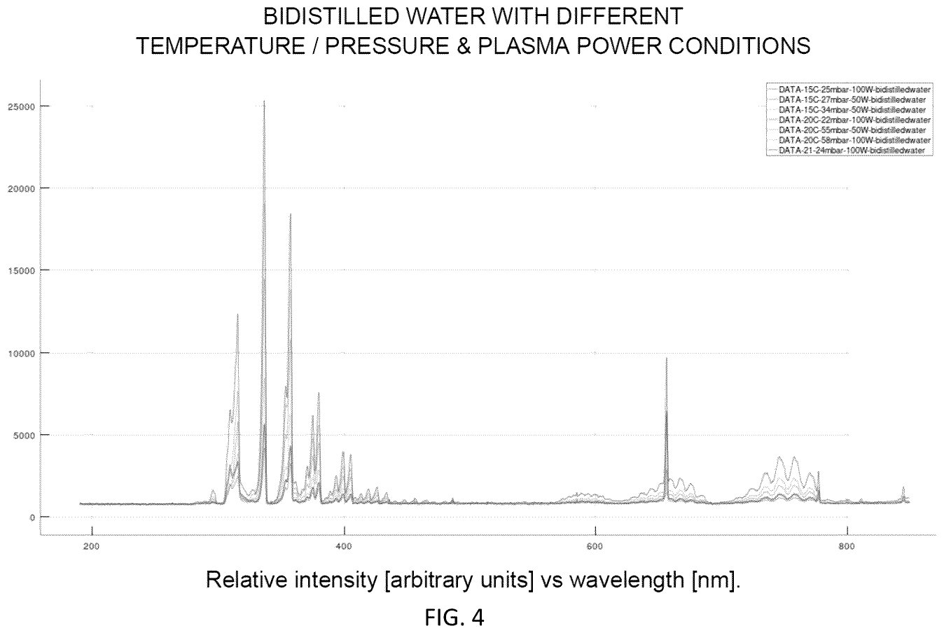

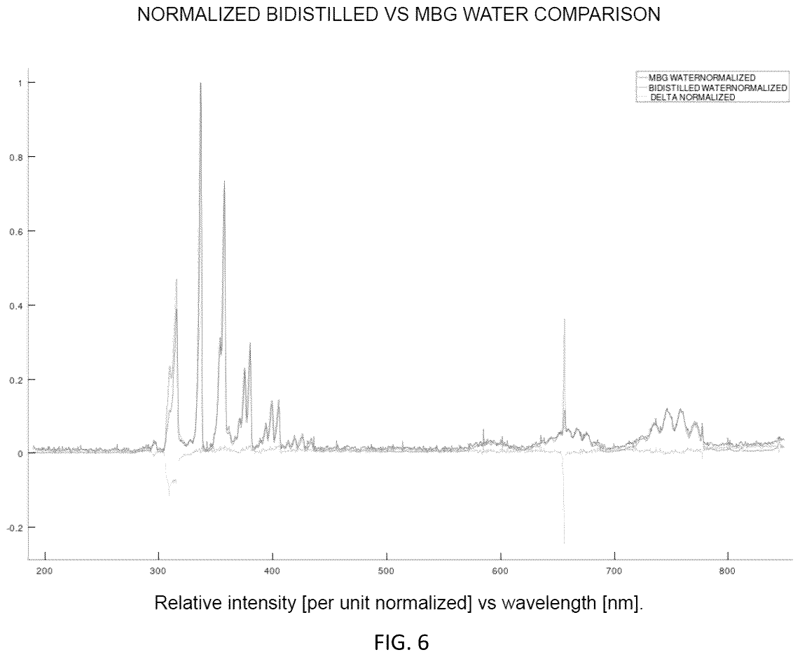

FIG. 4 illustrates a chart of intensity versus wavelength for analysis of bidistilled water at different temperatures and pressures generated by the present invention. FIG. 5 illustrates a chart of intensity versus wavelength for analysis of molecular biology grade (MBG) water at different temperatures and pressures generated by the present invention. FIG. 6 illustrates a chart of intensity versus wavelength, comparing normalized peaks of bidistilled and molecular biology grade (MBG) generated by the present invention. As shown in FIGS. 4-6, the device disclosed in the present invention has sufficient sensitivity to detect even different peaks between differently purified or ionized levels of water, demonstrating a high capability for detection of other molecules. For the purposes of analysis, the MBG water spectra was used as a baseline for detecting other molecules as discussed further below.

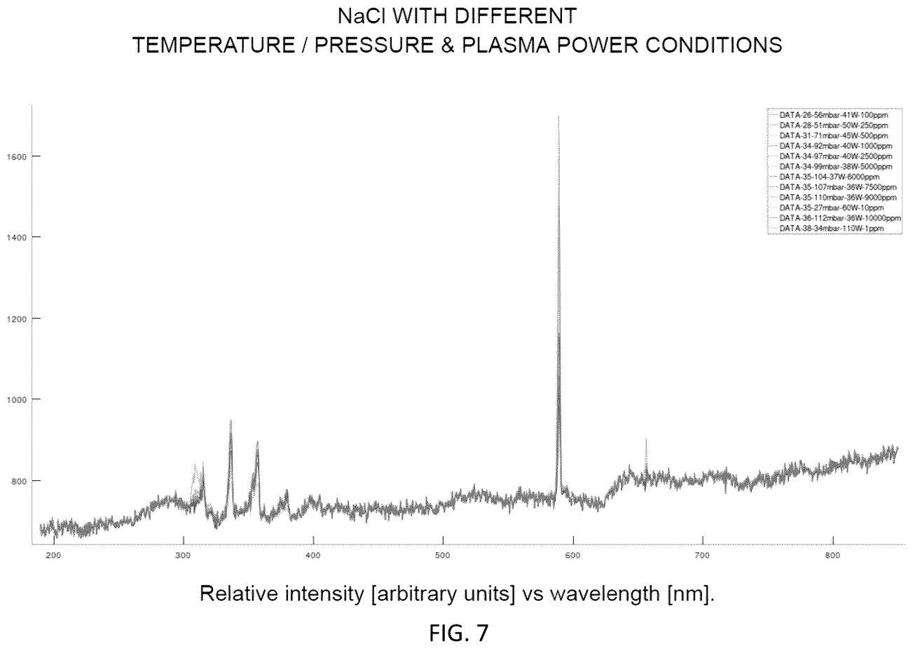

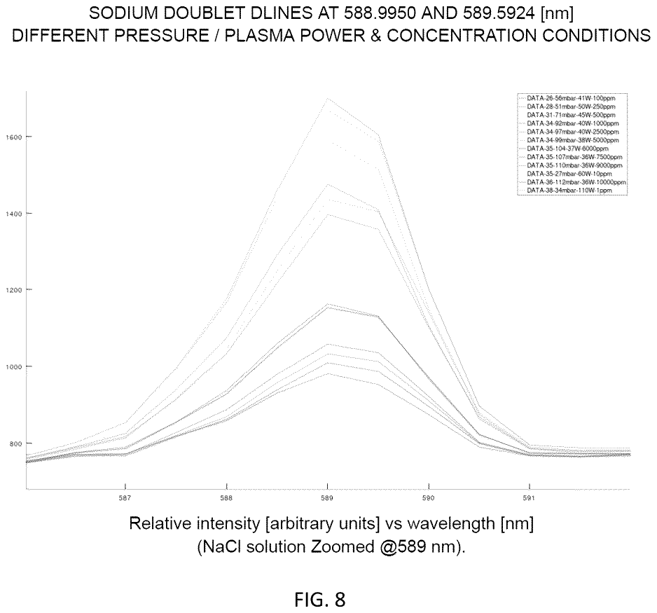

FIG. 7 illustrates a chart of intensity versus wavelength, comparing normalized peaks of sodium chloride (NaCl) at different concentrations, pressures, and plasma power conditions generated by the present invention. FIG. 8 illustrates a zoomed-in intensity versus wavelength chart for sodium chloride at different pressures, concentrations, and plasma power conditions, focused around a peak at 589 nm generated by the present invention. By checking the peak at different conditions, including concentrations, pressures, and plasma power conditions, the system demonstrating relatively consistent peaks in the spectrum, but with the peaks having different intensities depending on those conditions. The charts in FIGS. 7-8 demonstrate that, generally, higher peaks are detecting at higher concentrations, allowing the system to have an ability to distinguish amounts of certain chemicals, in addition to identify their presences based on characteristic peaks.

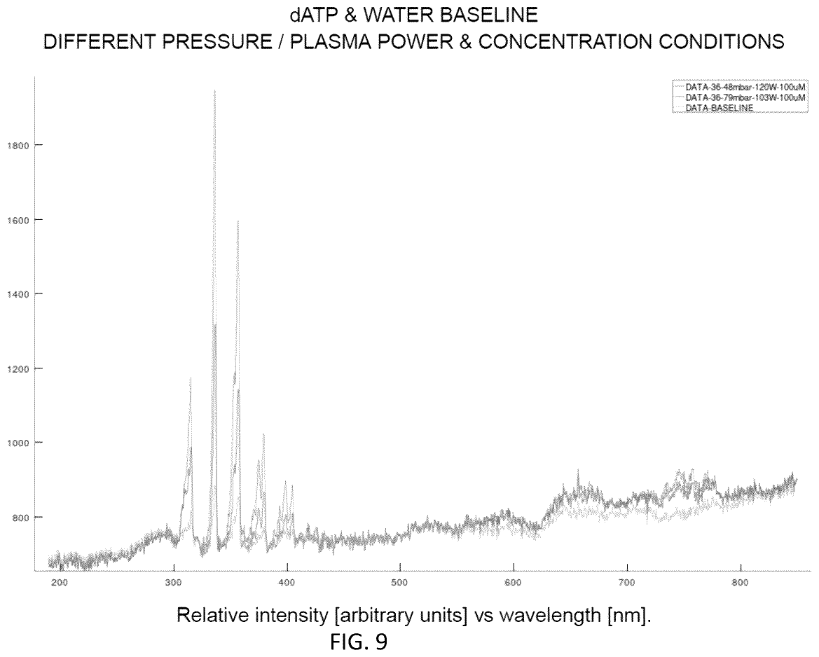

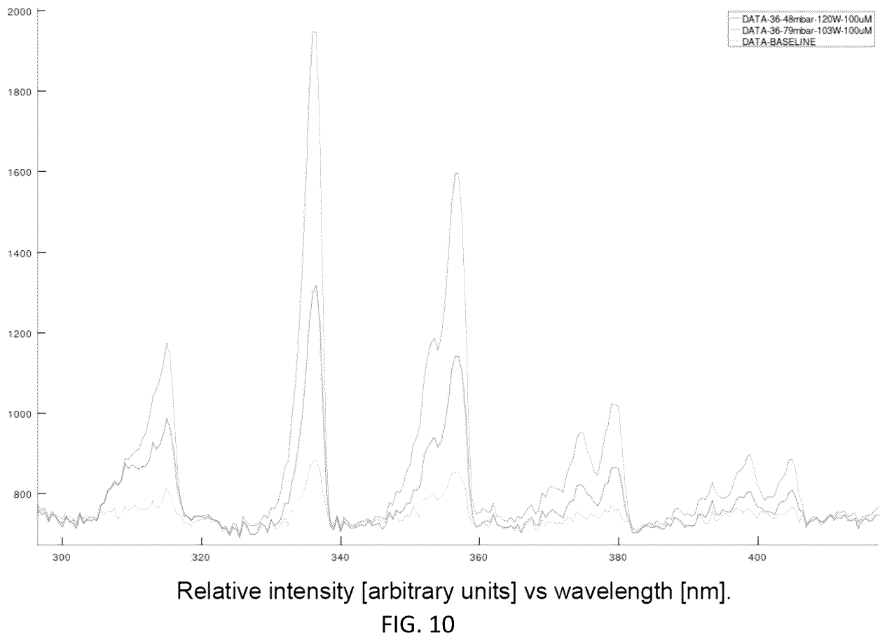

FIG. 9 illustrates a chart of intensity versus wavelength, comparing samples with dATP at different pressure, concentration, and plasma power conditions relative to a water baseline generated by the present invention. FIG. 10 illustrates a chart of intensity versus wavelength, comparing samples with dATP at different pressure, concentration, and plasma power conditions relative to a water baseline, zoomed in around 360 nm, generated by the present invention. FIGS. 9 and 10 show characteristic peaks that are higher than baseline peaks for the water sample, showing detection of the dATP.

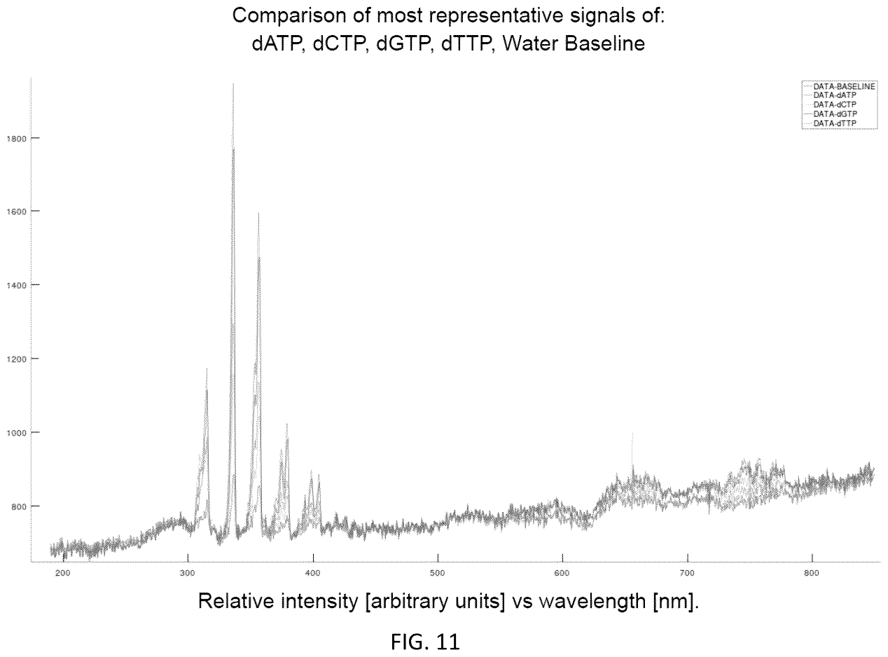

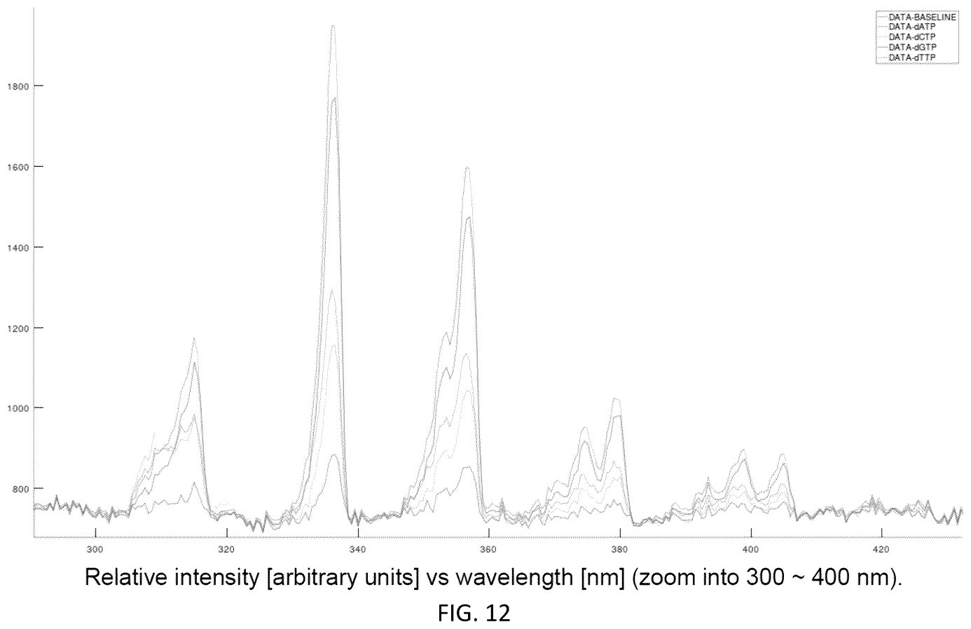

FIG. 11 illustrates a chart of intensity versus wavelength, comparing samples with dATP, dCTP, dGTP, dTTP relative to a water baseline generated by the present invention. FIG. 12 illustrates a chart of intensity versus wavelength, comparing samples with dATP, dCTP, dGTP, dTTP relative to a water baseline, zoomed in between 300 and 400 nm, generated by the present invention. FIGS. 11-12 demonstrate that the system is not only capable of detecting a compound present versus a water baseline, but also differentiation between different, but similar, compounds.

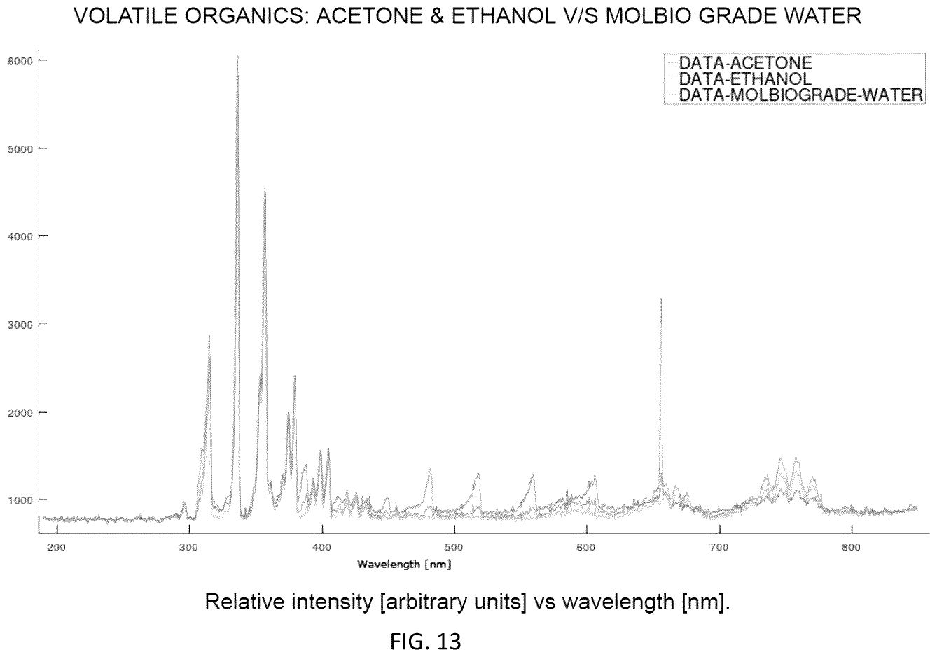

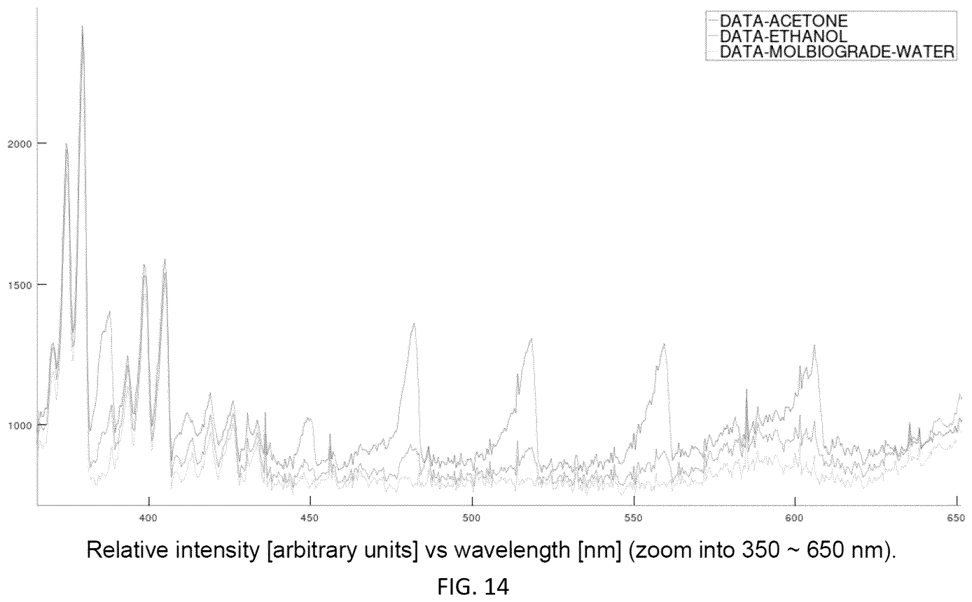

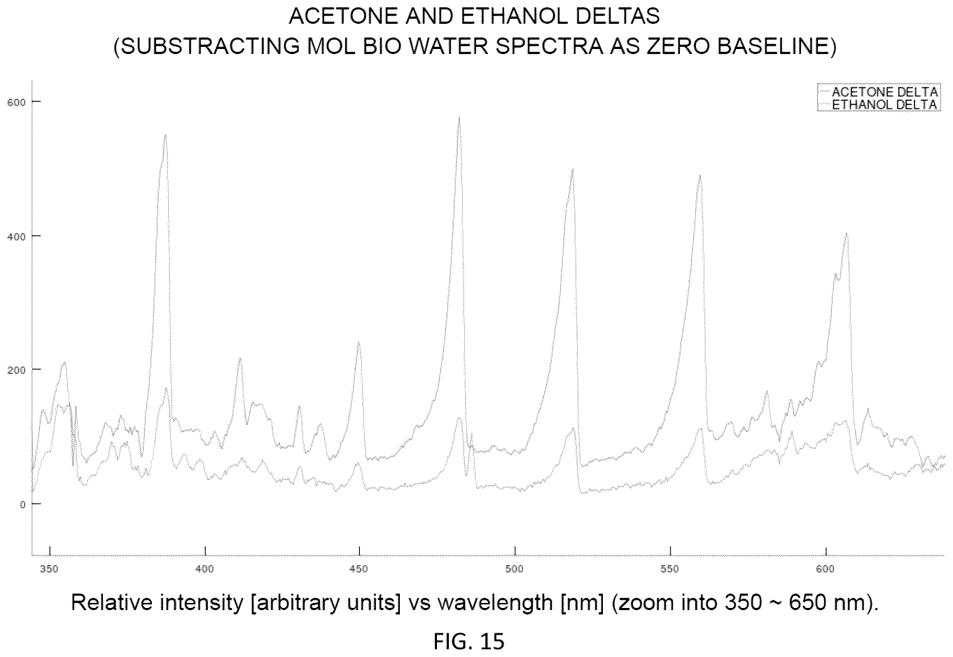

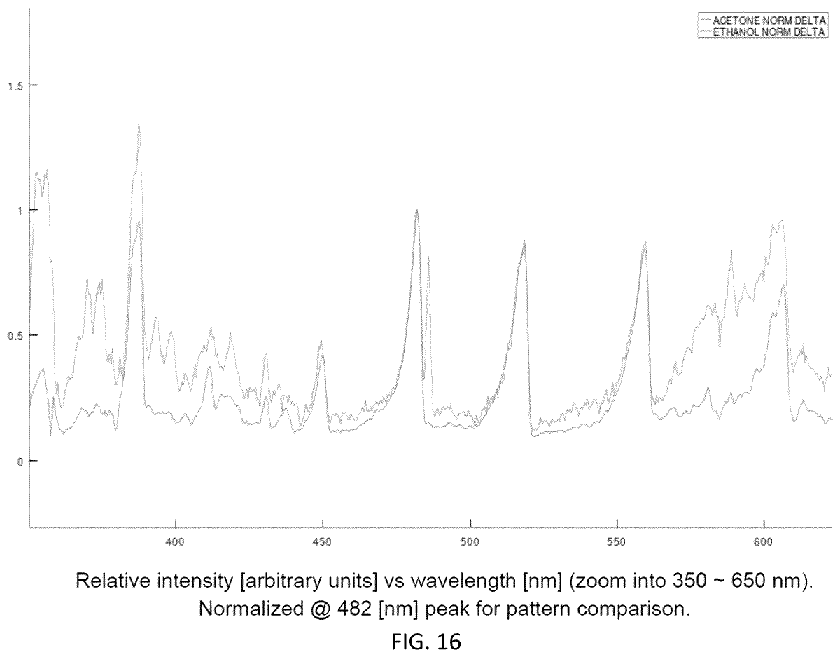

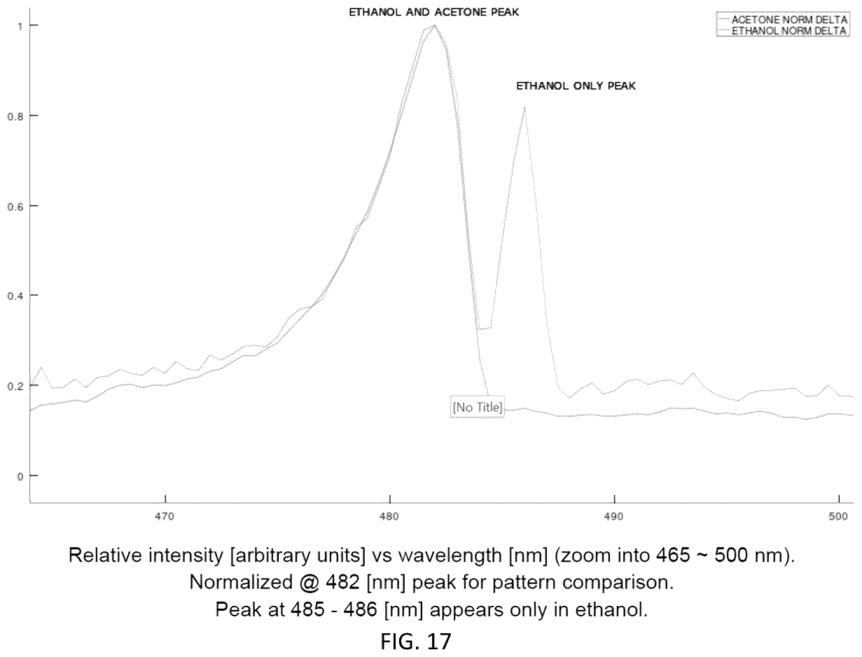

FIG. 13 illustrates a chart of intensity versus wavelength, comparing samples with acetone, ethanol, and MBG water generated by the present invention. FIG. 14 illustrates a chart of intensity versus wavelength, comparing samples with acetone, ethanol, and MBG water, zoomed in between 350 and 650 nm, generated by the present invention. FIG. 15 illustrates a chart of intensity versus wavelength, comparing samples with acetone and ethanol, zoomed in between 350 and 650 nm with the water baseline subtracted, generated by the present invention. FIG. 16 illustrates a chart of intensity versus wavelength, comparing samples with acetone and ethanol, zoomed in between 350 and 650 nm normalized at a peak at 482 nm, generated by the present invention. FIG. 17 illustrates a chart of intensity versus wavelength, comparing samples with acetone and ethanol, zoomed in between 465 and 500 nm, showing a peak unique to the ethanol signature at approximately 485 nm, generated by the present invention. FIGS. 13-17 demonstrate the present invention's ability to differentiate between different simple organic compounds, and particularly between different volatile organic compounds (VOCs).

In one embodiment, the optical emission spectrum covers at least 190 nm to 900 nm wavelengths where the analysis model utilizes both atomic emission lines and molecular band emissions.

In another embodiment, the present invention includes a system and method for analyzing a fluid sample within a reactor using non-thermal plasma excitation. In contrast to prior art continuous flow non-thermal plasma systems, the “no-flow” system of the present invention includes a reactor with valves controlling the movement of fluid into and out of the reactor.

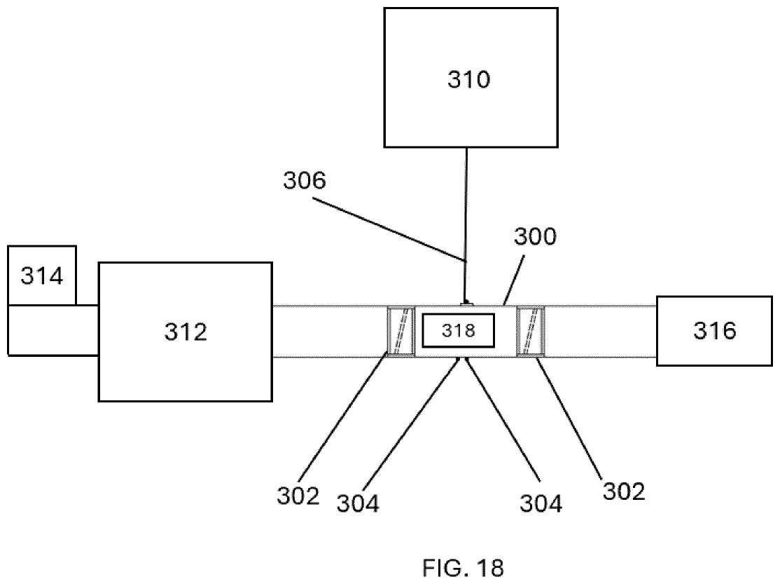

FIG. 18 illustrates a system with a plasma reactor including valves for static analysis of a fluid sample according to one embodiment of the present invention. The system includes a reactor 300 with valves 302 on either side of the reactor 300 which provide for isolating a fluid sample within the reactor 300. The fluid sample is operable to be introduced to the system through an inlet 314, nebulized by the nebulizer 312, enter the reactor 300, and exit the system through a vacuum pump 316 with a waste outlet after analysis. A plurality of electrodes 304 and an optical bridge 306 such as one or more quartz rods are in contact with the reactor 300 to provide for excitation of the sample and communication with a spectrometer 310, respectively. The valves 302 are actuated by a controller in communication with the valves 302, the vacuum pump 316 for pumping the fluid sample and a carrier into the reactor, and electrodes 304. In one embodiment, the controller is a wireless controller operable to send wireless signals to components within the system. In one embodiment, the controller includes a digital controller. The digital controller is operable to actuate the vacuum pump 316 to achieve a target pressure within the reactor 300, and to close a first valve 302 on an upstream side of the reactor and a second valve 302 on a downside side of the reactor such that the fluid sample is isolated within the reactor 300 and maintained at a target, constant pressure. In one embodiment, a pressure sensor 318 within the reactor which is in communication with the digital controller determines the target pressure has been reached. The valves 302 are operable to be butterfly valves or any other type of valve known in the art, including but not limited to electromagnetically actuated valves, quarter turn valves, and rising stem valves. Preferably, the valves 302 are operable to be actuated quickly and provide minimal fluidic resistance when in an open position. Certain valves which are suitable for the “no-flow” system of the present invention, such as butterfly valves, are disadvantageous for use in prior art continuous flow systems because these valves introduce turbulence into the system.

Once the target pressure is maintained, the fluid sample is hermetically sealed within the reactor 300 and the digital controller is operable to provide power to the electrodes 304 to generate non-thermal plasma from the fluid sample within the reactor 300. In one embodiment, the reactor 300 is a tube. Emissions from the fluid sample are operable to be transmitted through one or more optical bridges 306 between the reactor surface and a spectrometer. Preferred embodiments of such optical bridges for UV-VIS spectrometry include one or more quartz rods or UV-fused silica rods. However, other optical bridges are operable to be utilized depending on the type of spectrometer utilized, such as high-temperature resistant borosilicate glass for visible spectrometry and chalcogenide glass for infrared (IR) spectrometry.

Upon light emissions being transmitted through the optical bridge between the surface of the reactor 300 and the spectrometer 310, the spectrometer 310 records the temporal emission dynamics of the ionized molecules over the duration of a defined integration time. Different integration times are operable to be defined, and subsequent analyses of the fluid are operable to be conducted at different integration times. Time-resolved data is provided to an AI or ML module, which is operable to utilize the rate of spectral decay or shift as an additional data dimension to enhance the accuracy of analyte identification.

Any fluid sample described herein is operable to be analyzed by the “no-flow” system of the present invention. However, the present invention is particularly advantageous in analyzing complex fluid samples which are likely to contain molecules which have overlapping signatures, such as medical, industrial, biological, or environmental fluid samples. The present invention provides for distinguishing between complex, overlapping molecular signatures by monitoring the temporal evolution of the plasma-excited sample over variable integration times.

The intensity of elements in the fluid sample increase over time for some wavelengths. This provides another way to classify elements as the AI module is trained on data for increased intensity of certain elements over time as these elements are excited. This type of analysis is not possible in continuous flow spectrometry, as the fluid sample is not present in the reactor for a long enough time for an increased intensity to be observed. During excitation in the reactor, some compounds act as catalysts in causing other elements or compounds to emit free electrons, such as certain metals and hydrocarbons. In particular, it has been observed that some elements or compounds specifically increase the emission of other elements or compounds. As one example, potassium significantly increases emissions for hydroxyl groups. This causes a chain reaction or positive feedback loop within a closed environment, such as within the reactor chamber where the fluid sample and air are contained. Accordingly, the machine learning or artificial intelligence of the present invention is operable to be trained on known samples and the increased emissions caused by catalysts within the samples such as metals over time, providing another dimension for determining the compositions of samples which is not possible with prior art continuous flow systems because each molecule or compound enters and exits the reactor quickly in these continuous flow systems. The constrained environment within the reactor for the “no-flow” system of the present invention also provides for increased pressure and temperature compared to prior art continuous flow systems, thereby providing more excitement and more emissions.

The artificial intelligence or machine learning modules of the present invention are operable to generate custom formulas for the behavior of each element or molecule over time, including different variables such as pressure, temperature, intensity, and wavelength. By determining the values of constants, the present invention provides for identification and concentration determination of different elements or molecules. For example, some relationships are linear, whereas others are quadratic. For linear relationships, the present invention is operable to determine the slope for certain elements or molecules based on different variables and measurables. Some curves rise to the asymptotic level and flatten out over time, or reach the asymptotic level and then decrease. The artificial intelligence and/or machine learning modules of the present invention are operable to determine the best models for different variables for different elements, and provide constants for those models to enable identification which was not possible with prior art systems which do not provide for measurement of intensity over time and AI or ML analysis of the intensity over time for different elements or molecules.

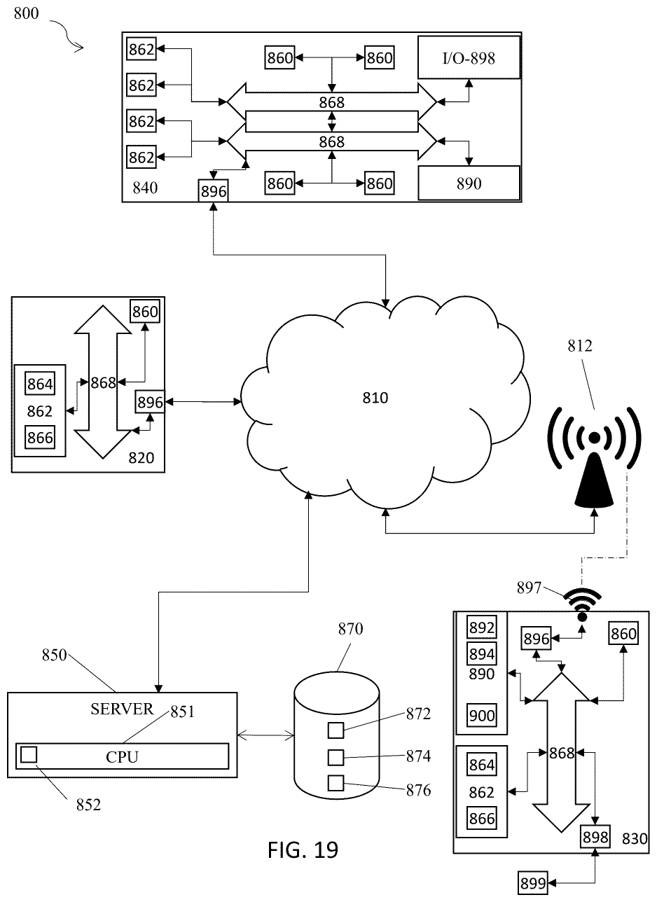

FIG. 19 is a schematic diagram of an embodiment of the invention illustrating a computer system, generally described as 800, having a network 810, a plurality of computing devices 820, 830, 840, a server 850, and a database 870.

The server 850 is constructed, configured, and coupled to enable communication over a network 810 with a plurality of computing devices 820, 830, 840. The server 850 includes a processing unit 851 with an operating system 852. The operating system 852 enables the server 850 to communicate through network 810 with the remote, distributed user devices. Database 870 is operable to house an operating system 872, memory 874, and programs 876.

In one embodiment of the invention, the system 800 includes a network 810 for distributed communication via a wireless communication antenna 812 and processing by at least one mobile communication computing device 830. Alternatively, wireless and wired communication and connectivity between devices and components described herein include wireless network communication such as WI-FI, WORLDWIDE INTEROPERABILITY FOR MICROWAVE ACCESS (WIMAX), Radio Frequency (RF) communication including RF identification (RFID), NEAR FIELD COMMUNICATION (NFC), BLUETOOTH including BLUETOOTH LOW ENERGY (BLE), ZIGBEE, Infrared (IR) communication, cellular communication, satellite communication, Universal Serial Bus (USB), Ethernet communications, communication via fiber-optic cables, coaxial cables, twisted pair cables, and/or any other type of wireless or wired communication. In another embodiment of the invention, the system 800 is a virtualized computing system capable of executing any or all aspects of software and/or application components presented herein on the computing devices 820, 830, 840. In certain aspects, the computer system 800 is operable to be implemented using hardware or a combination of software and hardware, either in a dedicated computing device, or integrated into another entity, or distributed across multiple entities or computing devices.

By way of example, and not limitation, the computing devices 820, 830, 840 are intended to represent various forms of electronic devices including at least a processor and a memory, such as a server, blade server, mainframe, mobile phone, personal digital assistant (PDA), smartphone, desktop computer, netbook computer, tablet computer, workstation, laptop, and other similar computing devices. The components shown here, their connections and relationships, and their functions, are meant to be exemplary only, and are not meant to limit implementations of the invention described and/or claimed in the present application.

In one embodiment, the computing device 820 includes components such as a processor 860, a system memory 862 having a random access memory (RAM) 864 and a read-only memory (ROM) 866, and a system bus 868 that couples the memory 862 to the processor 860. In another embodiment, the computing device 830 is operable to additionally include components such as a storage device 890 for storing the operating system 892 and one or more application programs 894, a network interface unit 896, and/or an input/output controller 898. Each of the components is operable to be coupled to each other through at least one bus 868. The input/output controller 898 is operable to receive and process input from, or provide output to, a number of other devices 899, including, but not limited to, alphanumeric input devices, mice, electronic styluses, display units, touch screens, gaming controllers, joy sticks, touch pads, signal generation devices (e.g., speakers), augmented reality/virtual reality (AR/VR) devices (e.g., AR/VR headsets), or printers.

By way of example, and not limitation, the processor 860 is operable to be a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and/or other manipulations of information.

In another implementation, shown as 840 in FIG. 19, multiple processors 860 and/or multiple buses 868 are operable to be used, as appropriate, along with multiple memories 862 of multiple types (e.g., a combination of a DSP and a microprocessor, a plurality of microprocessors, one or more microprocessors in conjunction with a DSP core).

Also, multiple computing devices are operable to be connected, with each device providing portions of the necessary operations (e.g., a server bank, a group of blade servers, or a multi-processor system). Alternatively, some steps or methods are operable to be performed by circuitry that is specific to a given function.

According to various embodiments, the computer system 800 is operable to operate in a networked environment using logical connections to local and/or remote computing devices 820, 830, 840 through a network 810. A computing device 830 is operable to connect to a network 810 through a network interface unit 896 connected to a bus 868. Computing devices are operable to communicate communication media through wired networks, direct-wired connections or wirelessly, such as acoustic, RF, or infrared, through an antenna 897 in communication with the network antenna 812 and the network interface unit 896, which are operable to include digital signal processing circuitry when necessary. The network interface unit 896 is operable to provide for communications under various modes or protocols.

In one or more exemplary aspects, the instructions are operable to be implemented in hardware, software, firmware, or any combinations thereof. A computer readable medium is operable to provide volatile or non-volatile storage for one or more sets of instructions, such as operating systems, data structures, program modules, applications, or other data embodying any one or more of the methodologies or functions described herein. The computer readable medium is operable to include the memory 862, the processor 860, and/or the storage media 890 and is operable be a single medium or multiple media (e.g., a centralized or distributed computer system) that store the one or more sets of instructions 900. Non-transitory computer readable media includes all computer readable media, with the sole exception being a transitory, propagating signal per se. The instructions 900 are further operable to be transmitted or received over the network 810 via the network interface unit 896 as communication media, which is operable to include a modulated data signal such as a carrier wave or other transport mechanism and includes any delivery media. The term “modulated data signal” means a signal that has one or more of its characteristics changed or set in a manner as to encode information in the signal.

Storage devices 890 and memory 862 include, but are not limited to, volatile and non-volatile media such as cache, RAM, ROM, EPROM, EEPROM, FLASH memory, or other solid state memory technology; discs (e.g., digital versatile discs (DVD), HD-DVD, BLU-RAY, compact disc (CD), or CD-ROM) or other optical storage; magnetic cassettes, magnetic tape, magnetic disk storage, floppy disks, or other magnetic storage devices; or any other medium that can be used to store the computer readable instructions and which can be accessed by the computer system 800.

In one embodiment, the computer system 800 is within a cloud-based network. In one embodiment, the server 850 is a designated physical server for distributed computing devices 820, 830, and 840. In one embodiment, the server 850 is a cloud-based server platform. In one embodiment, the cloud-based server platform hosts serverless functions for distributed computing devices 820, 830, and 840.

In another embodiment, the computer system 800 is within an edge computing network. The server 850 is an edge server, and the database 870 is an edge database. The edge server 850 and the edge database 870 are part of an edge computing platform. In one embodiment, the edge server 850 and the edge database 870 are designated to distributed computing devices 820, 830, and 840. In one embodiment, the edge server 850 and the edge database 870 are not designated for distributed computing devices 820, 830, and 840. The distributed computing devices 820, 830, and 840 connect to an edge server in the edge computing network based on proximity, availability, latency, bandwidth, and/or other factors.

It is also contemplated that the computer system 800 is operable to not include all of the components shown in FIG. 19, is operable to include other components that are not explicitly shown in FIG. 19, or is operable to utilize an architecture completely different than that shown in FIG. 19. The various illustrative logical blocks, modules, elements, circuits, and algorithms described in connection with the embodiments disclosed herein are operable to be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application (e.g., arranged in a different order or partitioned in a different way), but such implementation decisions should not be interpreted as causing a departure from the scope of the present invention.

Certain modifications and improvements will occur to those skilled in the art upon a reading of the foregoing description. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the present invention.

1-15. (canceled)

16. A method for identifying elements, molecules, and/or mixtures in a sample, comprising:
isolating a fluid sample within a reactor by closing a first valve on a first side of the reactor and a second valve on a second side of the reactor;
applying power to a plurality of electrodes surrounding the reactor to create a non-thermal plasma within the fluid sample;
transmitting optical emissions via at least one optical bridge to a spectrometer;
generating time-resolved spectral data for the fluid sample over a target integration time; and
processing the time-resolved spectral data to determine presence and/or concentration of one or more analytes in the fluid sample.

17. The method of claim 16, wherein processing the time-resolved spectral data includes using an artificial intelligence (AI) model or machine learning (ML) model.

18. The method of claim 17, wherein the AI model or ML model is operable to utilize temporal changes in the time-resolved spectral data to determine the presence and/or concentration of the one or more analytes in the fluid sample.

19. The method of claim 16, wherein the spectrometer is a UV-VIS spectrometer.

20. The method of claim 16, wherein closing the first valve and the second valve prevents the fluid sample from leaving the reactor, and wherein the fluid sample is maintained at a substantially constant pressure within the reactor when the non-thermal plasma is created within the fluid sample.
